(19) United States
(12) Patent Application Publication
(10) Pub. No.: US 2024/0199585 A1
(43) Pub. Date: Jun. 20, 2024

(54) SOLID FORMS OF (3R)-N-[2-CYANO-4-FLUORO-3-(3-METHYL-4-OXO-QUINAZOLIN-6-YL)OXY-PHENYL]-3-FLUORO-PYRROLIDINE-1-SULFONAMIDE

(71) Applicant: Hoffmann-La Roche Inc., Little Falls, NJ (US)

(72) Inventors: Bengt ÅSLUND, Stockholm (SE); Manuel KONRATH, Basel (CH); Ulf Goeran LARSSON, Stockholm (SE); Johan Salman MALMBERG, Stockholm (SE); Alexander Bogdan Emil MINIDIS, Stockholm (SE); Isabelle Georgette Huguette MODOLO-CHELLAT, Riehen (CH)

(73) Assignee: Hoffmann-La Roche Inc., Little Falls, NJ (US)

(21) Appl. No.: 18/533,710

(22) Filed: Dec. 8, 2023

Related U.S. Application Data
(63) Continuation of application No. PCT/EP2022/065342, filed on Jun. 7, 2022.

(30) Foreign Application Priority Data
Jun. 9, 2021 (EP) 21178622.3

(51) Int. Cl. C07D 403/12; A61K 9/00; A61K 9/02; A61K 9/20; A61K 9/48; A61K 31/517; A61K 47/10; A61K 47/12; A61K 47/26; A61K 47/32; A61K 47/38

(52) U.S. Cl. CPC C07D 403/12; A61K 9/0019; A61K 9/009; A61K 9/02; A61K 9/2013; A61K 9/2018; A61K 9/2054; A61K 9/2059; A61K 9/4825; A61K 9/4858; A61K 9/4866; A61K 9/4875; A61K 31/517; A61K 47/10; A61K 47/12; A61K 47/26; A61K 47/32; A61K 47/38

(57) ABSTRACT

The present invention provides solid forms of (3R)-N-[2-cyano-4-fluoro-3-(3-methyl-4-oxo-quinazolin-6-yl)oxy-phenyl]-3-fluoro-pyrrolidine-1-sulfonamide and solvates thereof, as well as therapeutic uses and processes to manufacture the new solid forms.

### CROSS-REFERENCE TO RELATED APPLICATIONS

This application is a Continuation of International Application No. PCT/EP2022/065342, filed on Jun. 7, 2022, which claims benefit of priority to European Application No. 21178622.3, filed on Jun. 9, 2021, each of which is incorporated herein by reference in its entirety

### FIELD OF THE INVENTION

The present invention provides new solid forms of (3R)-N-[2-cyano-4-fluoro-3-(3-methyl-4-oxo-quinazolin-6-yl)oxy-phenyl]-3-fluoro-pyrrolidine-1-sulfonamide, as well as therapeutic uses and processes to manufacture the new solid forms.

### BACKGROUND

The Rapidly Accelerated Fibrosarcoma (RAF) class of serine-threonine kinases comprise three members (ARAF, BRAF, RAF1) that compose the first node of the MAP kinase signalling pathway. Despite the apparent redundancy of the three RAF isoforms in signalling propagation through phosphorylation of MEK1 and 2, frequent oncogenic activating mutations are commonly found only for BRAF. In particular, substitution of V600 with glutamic acid or lysine renders the kinase highly activated with consequent hyper-stimulation of the MAPK pathway, independently from external stimulations (Cell. 2015 June 18; 161(7): 1681-1696).

Mutant BRAF is a targetable oncogenic driver and three BRAF inhibitors (vemurafenib, dabrafenib and encorafenib) reached the market up to now showing efficacy in BRAFV600E-positive melanoma. However rapid acquisition of drug resistance is almost universally observed and the duration of the therapeutic benefits for the targeted therapy remains limited.

Moreover, the developed BRAF inhibitors revealed an unexpected and “paradoxical” ability to repress MAPK signalling in BRAFV600E-driven tumours while the same inhibitors presented MAPK stimulatory activities in BRAF wild type (WT) models (N Engl J Med 2012; 366:271-273; and British Journal of Cancer volume 111, pages 640-645(2014)).

Mechanistic studies on the RAF paradox then clarified that oncogenic BRAFV600E phosphorylates MEK 1/2 in its monomeric cytosolic form while WT BRAF and RAF1 activation requires a complex step of events including cell membrane translocation and homo and/or heterodimerization promoted by activated RAS (KRAS, NRAS, HRAS) (Nature Reviews Cancer volume 14, pages 455-467(2014)).

The binding of inhibitors like vemurafenib, dabrafenib or encorafenib to a WT BRAF or RAF1 protomer, quickly induces RAF homo and/or hetero dimerization and membrane association of the newly formed RAF dimer. In the dimeric conformation, one RAF protomer allosterically induces conformational changes of the second resulting in a kinase active status and, importantly, in a conformation unfavourable for the binding of the inhibitor. The dimer induced by drug treatment, as a result, promotes MEK phosphorylation by the catalysis operated by the unbound protomer with hyperactivation of the pathway.

The RAF paradox results in two clinically relevant consequences: 1) accelerated growth of secondary tumours upon BRAFi monotherapy (mainly keratochantoma and squamous-cell carcinomas) (N Engl J Med 2012; 366:271-273) and 2) the acquisition of drug resistance in the setting of BRAFi monotherapy as well as in combinations of BRAFi+MEKi presents activation of dimer-mediated RAF signalling by genetically driven events including RAS mutations, BRAF amplifications, expression of dimeric-acting BRAF splice variants (Nature Reviews Cancer volume 14, pages 455-467(2014)). There is thus the need for RAF inhibitors capable of breaking that paradox.

Furthermore, the currently approved classical BRAF inhibitors Vemurafenib (Mol. Pharmaceutics 2012, 9, 11, 3236-3245), Dabrafenib (J Pharmacol Ex Ther 2013, 344 (3) 655-664) and Encorafenib (Pharmacol Res. 2018; 129:414-423) all have very poor brain permerability. This is major limitation for the use of those classical BRAF inhibitors for the treatment of brain cancer or brain metastases. There is thus the need for BRAF inhibitors having improved brain permeability.

There is accordingly a need for compounds that are efficient BRAF inhibitors showing considerably less paradoxial activation of the MAPK signaling pathway while retaining high potency. Such compounds can be referred to as a paradox breaker or RAF paradox breaker, in contrast to compounds inducing the RAF paradox (and which could be referred to as paradox inducers or RAF paradox inducers). (3R)-N-[2-cyano-4-fluoro-3-(3-methyl-4-oxo-quinazolin-6-yl)oxy-phenyl]-3-fluoro-pyrrolidine-1-sulfonamide satisfies these needs, and is a paradox breaking BRAF inhibitor with favourable brain penetration properties.

### SUMMARY OF THE DISCLOSURE

Polymorphs are different crystalline forms of the same compound. Polymorphs typically have a different crystal structure due to a different packing of the molecules in the lattice. Polymorphic forms are of interest to the pharmaceutical industry and especially to those involved in the development of suitable dosage forms. If the polymorphic form is not held constant during clinical studies, the exact dosage form used or studie may not be comparable form one lot to another. It is also desirable to have processes for producing a compound with the selected polymorphic form in high purity when the compound is used in clinical studies or commercial products since any impurities may produce undesired effects (e.g. toxicity). Certain polymorphs may display may also exhibit enhanced stability or may be more readily manufactured in high purity in large quantities, and are more suitable for inclusion in pharmaceutical formulations. Certain polymorphs may display other advantageous physical properties such as lack of hygroscopic tendencies, improved solubility, and enhanced rates of dissolution due to different lattic energies.

(3R)-N-[2-cyano-4-fluoro-3-(3-methyl-4-oxo-quinazolin-6-yl)oxy-phenyl]-3-fluoro-pyrrolidine-1-sulfonamide is a pardox breaking BRAF inhibitor with favourable brain penetration properties and useful in the therapy of cancer, in particular melanoma, lung cancer and brain metastatic cancer. Accordingly, for pharmaceutical development and commercialization, there is a need to identify solid forms of (3R)-N-[2-cyano-4-fluoro-3-(3-methyl-4-oxo-quinazolin-6-yl)oxy-phenyl]-3-fluoro-pyrrolidine-1-sulfonamide having desirable properties such as high crystallinity, high purity, and favourable physical stability, chemical stability, dissolution and mechanical properties. The present invention provides (3R)-N-[2-cyano-4-fluoro-3-(3-methyl-4-oxo-quinazolin-6-yl)oxy-phenyl]-3-fluoro-pyrrolidine-1-sulfonamide in two distinct solid forms: (i) a crystalline polymorphic form A and (ii) an amorphous form.

### BRIEF DESCRIPTION OF THE SEVERAL VIEWS OF THE DRAWINGS

FIG. 1 illustrates a X-ray powder diffraction pattern of the polymorphic Form A of (3R)-N-[2-cyano-4-fluoro-3-(3-methyl-4-oxo-quinazolin-6-yl)oxy-phenyl]-3-fluoro-pyrrolidine-1-sulfonamide.

FIG. 2 illustrates a X-ray powder diffraction pattern of amorphous (3R)-N-[2-cyano-4-fluoro-3-(3-methyl-4-oxo-quinazolin-6-yl)oxy-phenyl]-3-fluoro-pyrrolidine-1-sulfonamide.

FIG. 3 is a thermogram of the polymorphic form A of (3R)-N-[2-cyano-4-fluoro-3-(3-methyl-4-oxo-quinazolin-6-yl)oxy-phenyl]-3-fluoro-pyrrolidine-1-sulfonamide obtained by differential scanning calorimetry (DSC). A sharp melting signal was observed (onset 216.1° C., peak 217.6° C., enthalpy 98.3 J/g). A thermogram of the polymorphic form A of (3R)-N-[2-cyano-4-fluoro-3-(3-methyl-4-oxo-quinazolin-6-yl)oxy-phenyl]-3-fluoro-pyrrolidine-1-sulfonamide was also obtained by thermogravimetric analysis (TGA). No significant massloss was observed. A sharp melting signal as observed in DSC and decomposition occurs after melting.

FIG. 4 is a thermogram of amorphous (3R)-N-[2-cyano-4-fluoro-3-(3-methyl-4-oxo-quinazolin-6-yl)oxy-phenyl]-3-fluoro-pyrrolidine-1-sulfonamide obtained by differential scanning calorimetry (DSC). The observed thermal events are glass transition (onset 66.1° C., enthalpy 0.319 J/g), recrystallization (onset 117.4° C., Peak 123.5° C., enthalpy 66.1 J/g) and melting with decomposition (onset 209.1° C.).

FIG. 5 is a sorption/desorption curve of the polymorphic form A of (3R)-N-[2-cyano-4-fluoro-3-(3-methyl-4-oxo-quinazolin-6-yl)oxy-phenyl]-3-fluoro-pyrrolidine-1-sulfonamide obtained by dynamic vapour sorption (DVS). Masschange is <0.2% from 0%-90% RH; Polymorphic form A is not hygroscopic, no solid form change curing the cycle.

FIG. 6 is a sorption/desorption curve of amorphous (3R)-N-[2-cyano-4-fluoro-3-(3-methyl-4-oxo-quinazolin-6-yl)oxy-phenyl]-3-fluoro-pyrrolidine-1-sulfonamide obtained by dynamic vapour sorption (DVS). Masschange ˜3.8% from 0%-90% RH; the amorphous form is hygroscopic. Recrystallization to Form A observed in high humidity storage experiments.

FIG. 7 is a raman spectrum of the polymorphic form A of (3R)-N-[2-cyano-4-fluoro-3-(3-methyl-4-oxo-quinazolin-6-yl)oxy-phenyl]-3-fluoro-pyrrolidine-1-sulfonamide.

FIG. 8 is a raman spectrum of amorphous (3R)-N-[2-cyano-4-fluoro-3-(3-methyl-4-oxo-quinazolin-6-yl)oxy-phenyl]-3-fluoro-pyrrolidine-1-sulfonamide.

FIG. 9 is a IR spectrum of the polymorphic form A of (3R)-N-[2-cyano-4-fluoro-3-(3-methyl-4-oxo-quinazolin-6-yl)oxy-phenyl]-3-fluoro-pyrrolidine-1-sulfonamide.

FIG. 10 is a IR spectrum of amorphous (3R)-N-[2-cyano-4-fluoro-3-(3-methyl-4-oxo-quinazolin-6-yl)oxy-phenyl]-3-fluoro-pyrrolidine-1-sulfonamide.

### DETAILED DESCRIPTION OF THE DISCLOSURE

The present invention relates to a solid form of a compound of formula (I)

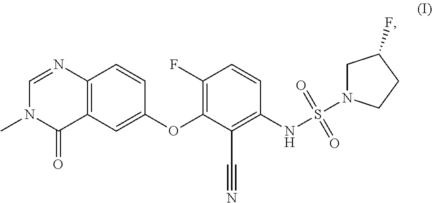 [SMILES: Cn1cnc2ccc(Oc3c(F)ccc(NS(=O)(=O)N4CC[C@@H](F)C4)c3C#N)cc2c1=O]

wherein the solid form is crystalline polymorph Form A or amorphous form.

The compound of formula (I) is also referred to as (3R)-N-[2-cyano-4-fluoro-3-(3-methyl-4-oxo-quinazolin-6-yl)oxy-phenyl]-3-fluoro-pyrrolidine-1-sulfonamide.

Crystalline polymorphic Form A is the thermodynamically stable form of (3R)-N-[2-cyano-4-fluoro-3-(3-methyl-4-oxo-quinazolin-6-yl)oxy-phenyl]-3-fluoro-pyrrolidine-1-sulfonamide. The crystalline polymorphic Form A is characterized by favourable biophysical properties such as for instance stability, solubility and is non-hygroscopic.

Amorphous (3R)-N-[2-cyano-4-fluoro-3-(3-methyl-4-oxo-quinazolin-6-yl)oxy-phenyl]-3-fluoro-pyrrolidine-1-sulfonamide is hygroscopic and is characterized by enhanced biophysical properties such as for instance solubility or bioavailability.

The terms “pharmaceutically acceptable carrier” and “pharmaceutically acceptable auxiliary substance” refer to carriers and auxiliary substances such as diluents or excipients that are compatible with the other ingredients of the formulation.

The term “room temperature” refers to 18-30° C., in particular 20-25° C., more particular to 20° C.

The terms “about” and “approximately” are interchangeably and refer to a range of values that fall within 5%, greater or less than the stated reference value. More particularly “about” or “approximately” refers to ±0.2° degrees 2-theta or ±0.5° C.

The terms “substantial amounts” as used herein, can mean at least 50%, in particular at least 60%, and more particular at least 70% of the initially present amount of a specific substance in a defined fraction. For instance after a purification step, the fraction comprising a substantial amount of a specific substance will comprise at least 50%, in particular at least 60%, more particularly at least 70% of the specific substance of the initially present amount of that specific substance prior to the purification step.

“Crystallization” and “recrystallization” may be used interchangeably; referring to a process that leads to a stable polymorph or crystalline form of a particular chemical compound wherein the chemical compound prior to the process can be in amorphous form, or dissolved or suspended in a solvent system. For example, the crystallization steps can be done by forming a crystal with a solvent and an anti-solvent.

“XRPD” refers the analytical method of X-Ray Powder Diffraction. The repeatability of the angular values is in the range of 2-theta ±0.2°. The term “approximately” given in combination with an angular value denotes the repeatability which is in the range of 2-theta The relative XRPD peak intensity is dependent upon many factors such as structure factor, temperature factor, crystallinity, polarization factor, multiplicity, and Lorentz factor. Relative intensities may vary considerably from one measurement to another due to preferred orientation effects. According to USP 941 (US Pharmacopoeia, 37th Edition, General Chapter 941), relative intensities between two samples of the same material may vary considerably due to “preferred orientation” effects. Anisotropic materials adopting preferred orientation will lead to anisotropic distribution of properties such as modulus, strength, ductility, toughness, electrical conductivity, thermal expansion, etc., as described e. g. in Kocks U. F. et al. (Texture and Anisotropy: Preferred Orientations in Polycrystals and Their Effect on Materials Properties, Cambridge University Press, 2000). In XRPD but also Raman spectroscopy, preferred orientations cause a change in the intensity distribution. Preferred orientation effects are particularly pronounced with crystalline APIs of relatively large particle size.

“Characteristic peak” refers to the presence of the powder X-ray diffraction peak definitively identifies the (3R)-N-[2-cyano-4-fluoro-3-(3-methyl-4-oxo-quinazolin-6-yl)oxy-phenyl]-3-fluoro-pyrrolidine-1-sulfonamide as the referenced crystalline form (Form A). Typically, the powder X-ray diffraction analysis is conducted at ambient conditions in transmission geometry with a STOE STADI P diffractometer (Cu Kα₁ radiation, primary monochromator, silicon strip detector, angular range 3 to 42 degrees two-theta, approximately 30 minutes total measurement time). The samples (approximately 10 to 50 mg) are prepared between thin polymer films and are analyzed without further processing (e. g. grinding or sieving) of the substance.

“Polymorph” refers to crystalline forms having the same chemical composition but different spatial arrangements of the molecules, atoms, and/or ions forming the crystal. In general, reference throughout this specification will be to a polymorphic form of (3R)-N-[2-cyano-4-fluoro-3-(3-methyl-4-oxo-quinazolin-6-yl)oxy-phenyl]-3-fluoro-pyrrolidine-1-sulfonamide. The term “polymorphic form” as used herein may or may not include other crystalline solid state molecular forms including hydrates (e.g. bound water present in the crystalline structure) of the same compound. Polymorphs typically have a different crystal structure due to a different packing of the molecules in the lattice. This results in a different crystal symmetry and/or unit cell parameters which directly influences its physical properties such as the X-ray diffraction characteristics of crystals or powder.

“Amorphous” refers to solid materials that lack the long-range order that is characteristic of a crystalline solid.

The term “solvate” refers herein to a molecular complex comprising a compound of formula (I) and a stoichiometric or non-stoichiometric amount of one or more solvent molecules (e. g., ethanol). “Hydrate” refers herein to a solvate comprising a compound of formula (I) and a stoichiometric or non-stoichiometric amount of water.

The terms “pharmaceutically acceptable excipient”, pharmaceutically acceptable carrier” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents or lubricants used in formulating pharmaceutical products.

The term “pharmaceutical composition” encompasses a product comprising specified ingredients in pre-determined amounts or proportions, as well as any product that results, directly or indirectly, from combining specified ingredients in specified amounts. Particularly it encompasses a product comprising one or more active ingredients, and an optional carrier comprising inert ingredients, as well as any product that results, directly or indirectly, from combination, complexation or aggregation of any two or more of the ingredients, or from dissociation of one or more of the ingredients, or from other types of reactions or interactions of one or more of the ingredients.

The terms “pharmaceutically acceptable carrier” and “pharmaceutically acceptable auxiliary substance” refer to carriers and auxiliary substances such as diluents or excipients that are compatible with the other ingredients of the formulation.

“Therapeutically effective amount” means an amount that is effective to prevent, alleviate or ameliorate symptoms of disease or prolong the survival of the subject being treated.

The term “substantially pure” when used in reference to a solid form (3R)-N-[2-cyano-4-fluoro-3-(3-methyl-4-oxo-quinazolin-6-yl)oxy-phenyl]-3-fluoro-pyrrolidine-1-sulfonamide refers to said polymorph being >90% pure. The solid form of (3R)-N-[2-cyano-4-fluoro-3-(3-methyl-4-oxo-quinazolin-6-yl)oxy-phenyl]-3-fluoro-pyrrolidine-1-sulfonamide does not contain more than 10% of any other compound, in particular does not contain more than 10% of any other solid form of (3R)-N-[2-cyano-4-fluoro-3-(3-methyl-4-oxo-quinazolin-6-yl)oxy-phenyl]-3-fluoro-pyrrolidine-1-sulfonamide.

More particular, the term “substantially pure” when used in reference to a solid form of (3R)-N-[2-cyano-4-fluoro-3-(3-methyl-4-oxo-quinazolin-6-yl)oxy-phenyl]-3-fluoro-pyrrolidine-1-sulfonamide refers to said solid being >95% pure. The solid form of (3R)-N-[2-cyano-4-fluoro-3-(3-methyl-4-oxo-quinazolin-6-yl)oxy-phenyl]-3-fluoro-pyrrolidine-1-sulfonamide does not contain more than 5% of any other compound, in particular does not contain more than 5% of any other solid form (3R)-N-[2-cyano-4-fluoro-3-(3-methyl-4-oxo-quinazolin-6-yl)oxy-phenyl]-3-fluoro-pyrrolidine-1-sulfonamide.

Even more particular, the term “substantially pure” when used in reference to a solid form of (3R)-N-[2-cyano-4-fluoro-3-(3-methyl-4-oxo-quinazolin-6-yl)oxy-phenyl]-3-fluoro-pyrrolidine-1-sulfonamide refers to said solid form being >97% pure. The solid form of (3R)-N-[2-cyano-4-fluoro-3-(3-methyl-4-oxo-quinazolin-6-yl)oxy-phenyl]-3-fluoro-pyrrolidine-1-sulfonamide does not contain more than 3% of any other compound, in particular does not contain more than 3% of any other solid form of (3R)-N-[2-cyano-4-fluoro-3-(3-methyl-4-oxo-quinazolin-6-yl)oxy-phenyl]-3-fluoro-pyrrolidine-1-sulfonamide.

Most particular, the term “substantially pure” when used in reference to a solid form of (3R)-N-[2-cyano-4-fluoro-3-(3-methyl-4-oxo-quinazolin-6-yl)oxy-phenyl]-3-fluoro-pyrrolidine-1-sulfonamide refers to said polymorph being >99% pure. The solid form of (3R)-N-[2-cyano-4-fluoro-3-(3-methyl-4-oxo-quinazolin-6-yl)oxy-phenyl]-3-fluoro-pyrrolidine-1-sulfonamide does not contain more than 1% of any other compound, in particular does not contain more than 1% of any other solid form of (3R)-N-[2-cyano-4-fluoro-3-(3-methyl-4-oxo-quinazolin-6-yl)oxy-phenyl]-3-fluoro-pyrrolidine-1-sulfonamide.

Most particular, the term “substantially pure” when used in reference to a solid form of (3R)-N-[2-cyano-4-fluoro-3-(3-methyl-4-oxo-quinazolin-6-yl)oxy-phenyl]-3-fluoro-pyrrolidine-1-sulfonamide refers to said polymorph being >99.5% pure. The solid form of (3R)-N-[2-cyano-4-fluoro-3-(3-methyl-4-oxo-quinazolin-6-yl)oxy-phenyl]-3-fluoro-pyrrolidine-1-sulfonamide does not contain more than 1% of any other compound, in particular does not contain more than 1% of any other solid form of (3R)-N-[2-cyano-4-fluoro-3-(3-methyl-4-oxo-quinazolin-6-yl)oxy-phenyl]-3-fluoro-pyrrolidine-1-sulfonamide.

While the present invention has been described with reference to the specific embodiments thereof, it should be understood by those skilled in the art that various changes can be made and equivalents can be substituted without departing from the true spirit and scope of the invention. In addition, many modifications can be made to adapt a particular situation, material, composition of matter, process, process step or steps, to the objective spirit and scope of the present invention. All such modifications are intended to be within the scope of the claims appended hereto. All separate embodiments can be combined.

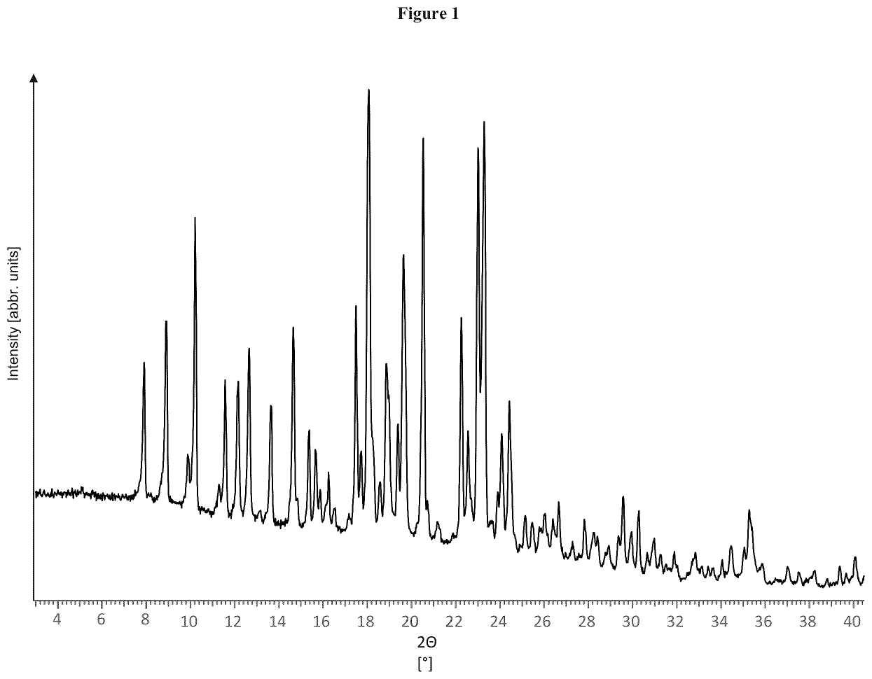

Aspects of the invention are:
A solid form of a compound of formula (I)

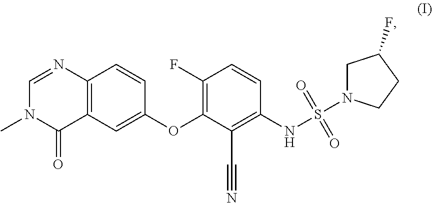 [SMILES: Cn1cnc2ccc(Oc3c(F)ccc(NS(=O)(=O)N4CC[C@@H](F)C4)c3C#N)cc2c1=O]

wherein the solid form is crystalline polymorphic Form A characterized by a X-ray powder diffraction pattern comprising a peak at an angle of diffraction at about 10.22 degrees 2-theta and at least one additional peak expressed in values of degrees 2-theta at approximately 7.90, 8.92, 11.58, 12.16, 12.66, 14.66, 17.50, 18.06, 19.64 or 20.54;
A solid form according to aspect 1, characterized by a X-ray powder diffraction pattern comprising a peak at an angle of diffraction at about 10.22 degrees 2-theta and at about 18.06 degrees 2-theta;
A solid form according to aspect 1, characterized by a X-ray powder diffraction pattern comprising a peak at an angle of diffraction at about 10.22 degrees 2-theta and at about 20.54 degrees 2-theta;
A solid form according to any one of aspects 1 to 3, characterized by a X-ray powder diffraction pattern comprising at least three of the peaks at an angle of diffraction at about 7.90, 8.92, 10.22, 11.58, 12.16, 12.66, 14.66, 17.50, 18.06, 19.64 and 20.54 degrees 2-theta;
A solid form according to any one of aspects 1 to 4, characterized by a X-ray powder diffraction pattern comprising the peaks at an angle of diffraction at about 7.90, 8.92, 10.22, 11.58, 12.16, 12.66, 14.66, 17.50, 18.06, 19.64 and 20.54 degrees 2-theta;
A solid form characterized by a X-Ray powder diffraction pattern according to any one of aspects 1 to 5, which is further comprising at least one additional peak expressed in values of degrees 2-theta at approximately 5.06, 9.88, 11.28, 13.16, 13.64, 14.84, 15.38, 15.66, 15.86, 16.24, 16.54, 17.18, 18.58, 18.98, 19.40, 20.72, 21.18, 22.26, 23.00, 23.30, 23.90, 24.08 or 24.44;
A solid form of a compound of formula (I) characterized by a X-Ray powder diffraction pattern according to the invention, wherein the pattern was obtained with CuKα1 radiation (1.5406 Å);
A solid form according to any one of aspects 1 to 7, characterized by the X-ray powder diffraction pattern as shown in FIG. 1;
A solid form according to any one of aspects 1 to 8, characterized by having a melting point with a peak signal at above 215° C., in particular between about 215.6° C. to about 219.6° C., using differential scanning calorimetry with a heating rate of 10 K/min;
A solid form of a compound of formula (I)

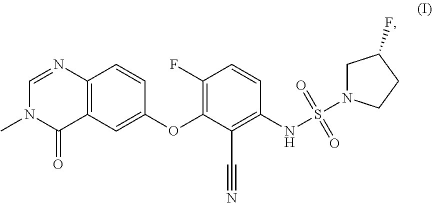 [SMILES: Cn1cnc2ccc(Oc3c(F)ccc(NS(=O)(=O)N4CC[C@@H](F)C4)c3C#N)cc2c1=O]

wherein the solid form is crystalline polymorphic Form A is characterized by having a melting point with a peak signal at above 215° C., in particular between about 215.6° C. to about 219.6° C., using differential scanning calorimetry with a heating rate of 10 K/min;
A solid form according to any one of aspects 1 to 10, wherein crystalline polymorphic Form A is further characterized by an IR spectrum comprising at least one peak at one of the positions 689 (±2) cm⁻¹, 1326 (±2) cm⁻¹ or 2874 (±2) cm⁻¹, in particular comprising at least two peaks at positions 689 (±2) cm⁻¹, 1326 (±2) cm⁻¹ or 2874 (±2) cm⁻¹, more particularly comprising the peaks at positions 689 (±2) cm⁻¹, 1326 (±2) cm⁻¹ and 2874 (±2) cm⁻¹;
A solid form of a compound of formula (I)

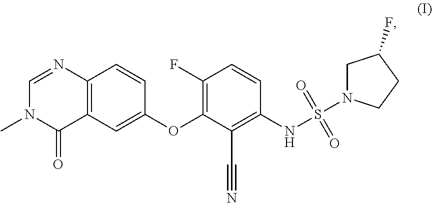 [SMILES: Cn1cnc2ccc(Oc3c(F)ccc(NS(=O)(=O)N4CC[C@@H](F)C4)c3C#N)cc2c1=O]

wherein the solid form is crystalline polymorphic Form A characterized by an IR spectrum comprising at least one peak at one of the positions 689 (±2) cm⁻¹, 1326 (±2) cm⁻¹ or 2874 (±2) cm⁻¹, in particular comprising at least two peaks at positions 689 (±2) cm⁻¹, 1326 (±2) cm⁻¹ or 2874 (±2) cm⁻¹, more particularly comprising the peaks at positions 689 (±2) cm⁻¹, 1326 (±2) cm⁻¹ and 2874 (±2) cm⁻¹;
A solid form according to any one of aspects 1 to 12, wherein the solid form is crystalline polymorphic Form A characterized by a Raman spectrum comprising at least one peak at one of the positions 691 (±2) cm⁻¹, 1660 (±2) cm⁻¹ or 3061 (±2) cm⁻¹, in particular comprising at least two peaks at positions 691 (±2) cm⁻¹, 1660 (±2) cm⁻¹ or 3061 (±2) cm⁻¹, more particularly comprising the peaks at positions 691 (±2) cm⁻¹, 1660 (±2) cm⁻¹ and 3061 (±2) cm⁻¹.

A solid form of a compound of formula (I)

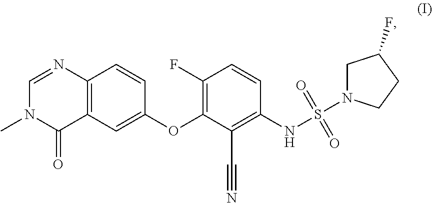 [SMILES: Cn1cnc2ccc(Oc3c(F)ccc(NS(=O)(=O)N4CC[C@@H](F)C4)c3C#N)cc2c1=O]

wherein the solid form is crystalline polymorphic Form A characterized by a Raman spectrum comprising at least one peak at one of the positions 691 (±2) cm⁻¹, 1660 (±2) cm⁻¹ or 3061 (±2) cm⁻¹, in particular comprising at least two peaks at positions 691 (±2) cm⁻¹, 1660 (±2) cm⁻¹ or 3061 (±2) cm⁻¹, more particularly comprising the peaks at positions 691 (±2) cm⁻¹, 1660 (±2) cm⁻¹ and 3061 (±2) cm⁻¹;
A solid form of a compound of formula (I) or a solvate thereof

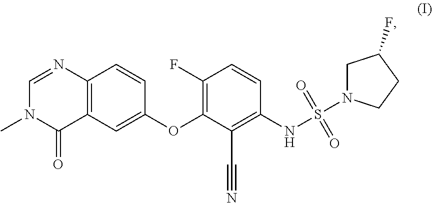 [SMILES: Cn1cnc2ccc(Oc3c(F)ccc(NS(=O)(=O)N4CC[C@@H](F)C4)c3C#N)cc2c1=O]

wherein the solid form is amorphous;
A solid form according to aspect 15, characterized by exhibiting an onset of a glass transition at a temperature of about 63.1° C. to about 69.1, in particular of about 64.6 to about 67.6, more particularly of about 66.1° C., using differential scanning calorimetry with a heating rate of 10 K/min;
A solid form according to aspect 15 or 16, characterized by exhibiting an onset of recrystallization at a temperature of about 114.4° C. to about 120.4° C., in particular between about 115.9° C. to about 118.9° C., more particularly of about 117.4° C. using differential scanning calorimetry with a heating rate of 10 K/min;
A solid form according to any one of aspects 15 to 17, wherein the solid form is characterized by an IR spectrum comprising at least one peak at one of the positions 679 (±2) cm⁻¹, 1035 (±2) cm⁻¹ or 1337 (±2) cm⁻¹, in particular comprising at least two peaks at positions 679 (±2) cm⁻¹, 1035 (±2) cm⁻¹ or 1337 (±2) cm⁻¹, more particular comprising the peaks at positions 679 (±2) cm⁻1, 1035 (±2) cm⁻¹ and 1337 (±2) cm⁻¹;
A solid form according to any one of aspects 15 to 18, wherein the solid form is characterized by a Raman spectrum comprising at least one peak at one of the positions 1159 (±2) cm⁻¹, 1344 (±2) cm⁻¹ or 3069 (±2) cm⁻¹, in particular comprising at least two peaks at positions 1159 (±2) cm⁻¹, 1344 (±2) cm⁻¹ or 3069 (±2) cm⁻¹, more particular having the peaks at positions 1159 (±2) cm⁻¹, 1344 (±2) cm⁻¹ and 3069 (±2) cm⁻¹;
A substantially pure solid form according to any one of aspects 1 to 19;
A solid form according to any one of aspects 1 to 20 for use as a medicament;
A solid form according to any one of aspects 1 to 20 for the treatment or prophylaxis of cancer, in particular BRAF associated cancer;
A solid form according to any one of aspects 1 to 20 for the treatment or prophylaxis of cancer, in particular melanoma or non-small cell lung cancer;
A pharmaceutical composition comprising a solid form according to any one of aspects 1 to 20 and one or more pharmaceutically acceptable auxiliary substances;
Use of a solid form according to the invention for the treatment of cancer, in particular BRAF associated cancer;
Use of a solid form according to the invention for the preparation of a medicament for the treatment of cancer, in particular BRAF associated cancer; and
A method for therapeutic or prophylactic treatment of cancer, in particular BRAF associated cancer, said method comprising administering an effective amount of a solid form according to the invention to a patient in need thereof.

In one embodiment the solid form of the crystalline polymorphic Form A of the compound of formula (I) according to the invention is a solvate.

A certain embodiment relates to the crystalline polymorphic Form A of the compound of formula (I) as described herein, characterized by the X-ray powder diffraction pattern as shown in FIG. 1.

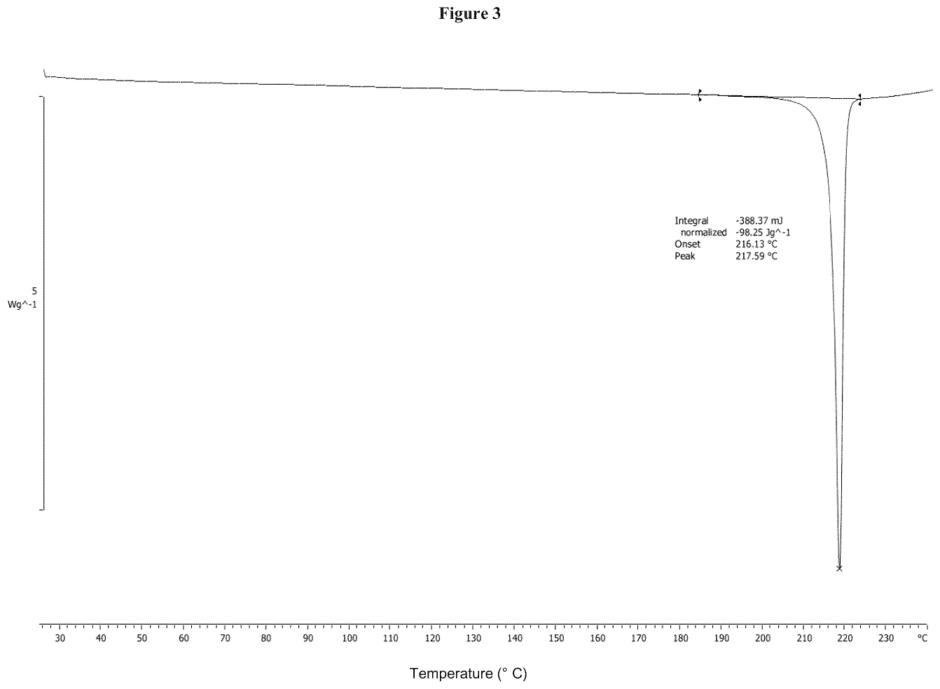

A certain embodiment relates to the crystalline polymorphic Form A of the compound of formula (I) as described herein, characterized by the differential scanning calorimetry thermogram as shown in FIG. 3.

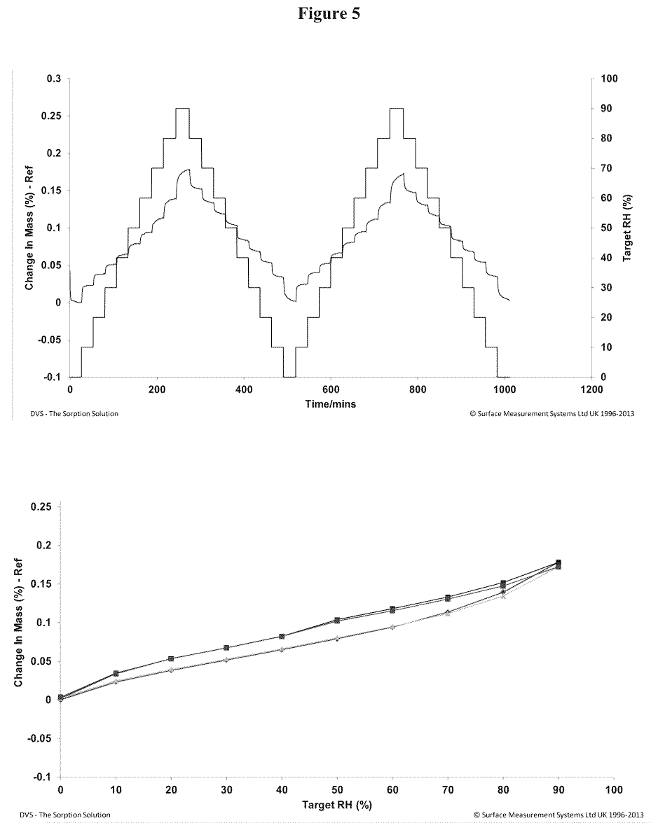

A certain embodiment relates to the crystalline polymorphic Form A of the compound of formula (I) as described herein, characterized by the dynamic vapour sorption profile as shown in FIG. 5.

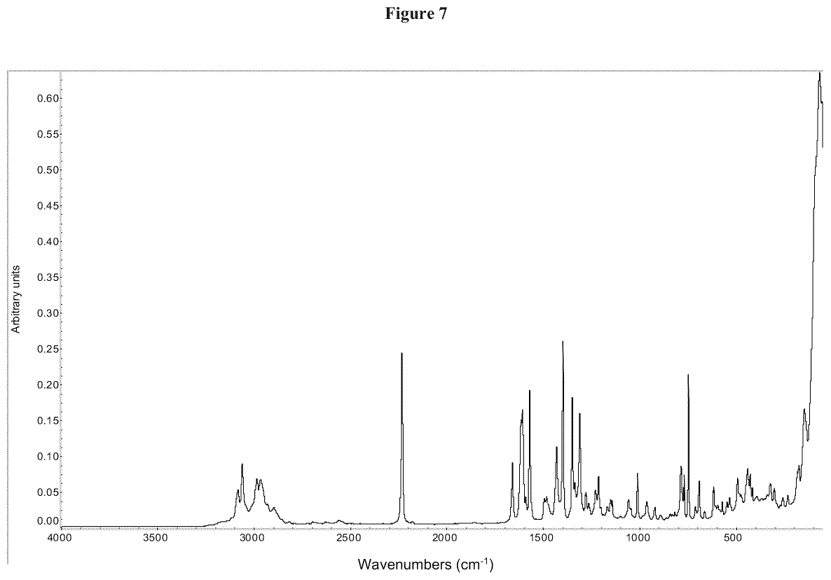

A certain embodiment relates to the crystalline polymorphic Form A of the compound of formula (I) as described herein, characterized by the raman spectrum as shown in FIG. 7.

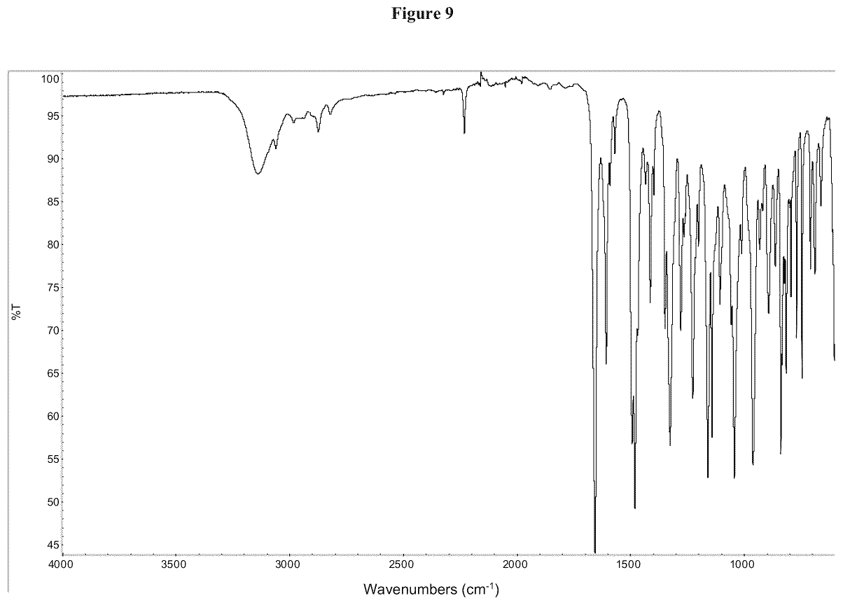

A certain embodiment relates to the crystalline polymorphic Form A of the compound of formula (I) as described herein, characterized by the IR spectrum as shown in FIG. 9.

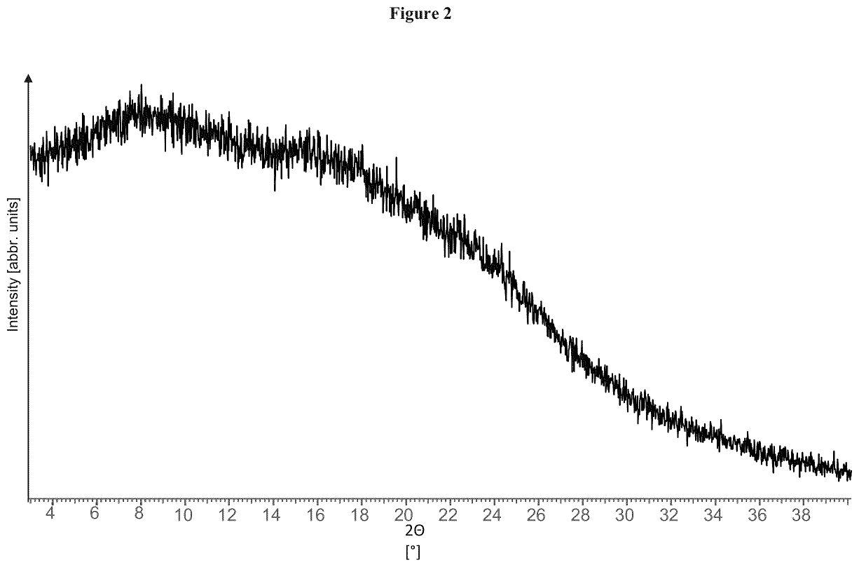

A certain embodiment relates to the amorpous form of the compound of formula (I) as described herein, characterized by the X-ray powder diffraction pattern as shown in FIG. 2.

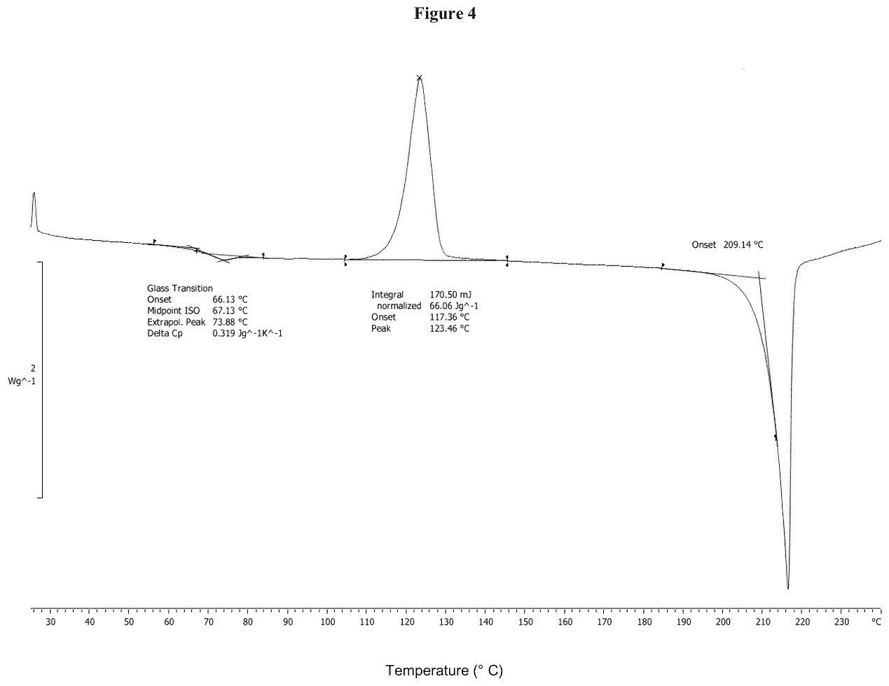

A certain embodiment to the amorpous form of the compound of formula (I) as described herein, characterized by the differential scanning calorimetry thermogram as shown in FIG. 4.

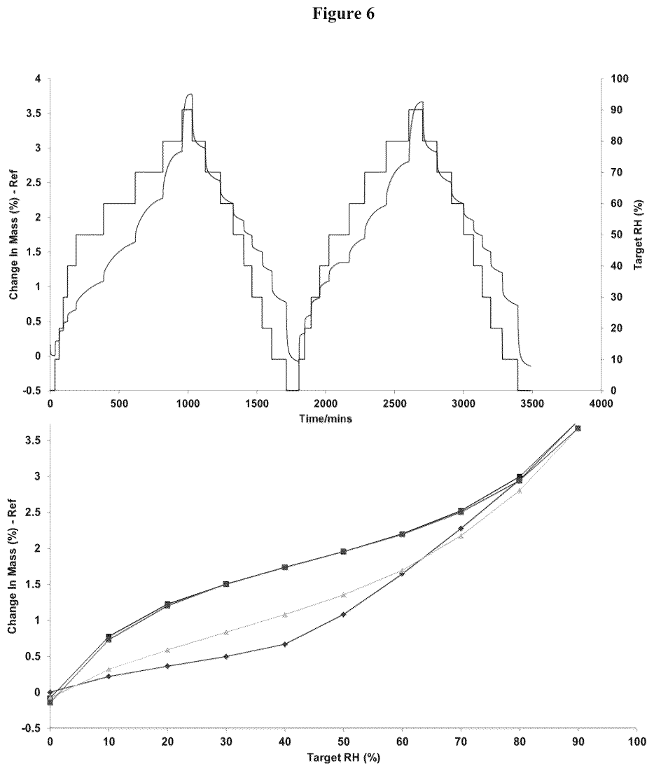

A certain embodiment relates to the amorpous form of the compound of formula (I) as described herein, characterized by the dynamic vapour sorption profile as shown in FIG. 6.

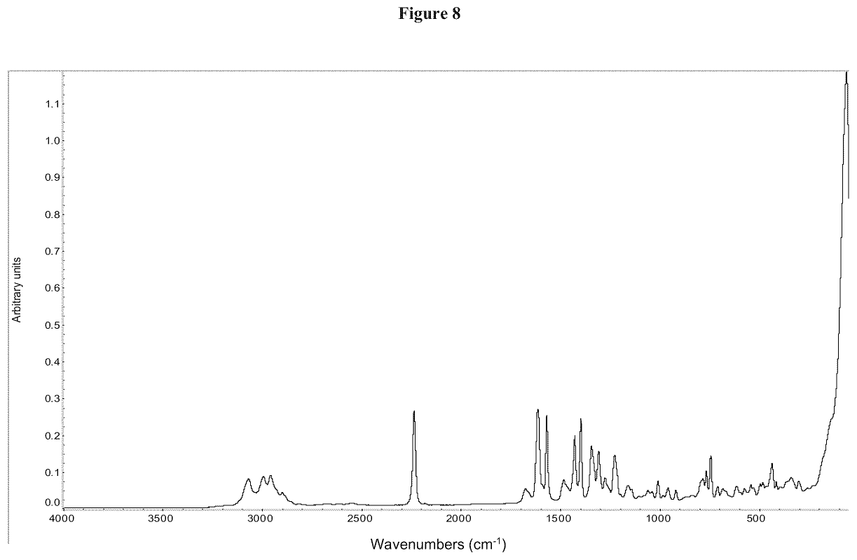

A certain embodiment relates to the amorpous form of the compound of formula (I) as described herein, characterized by the raman spectrum as shown in FIG. 8.

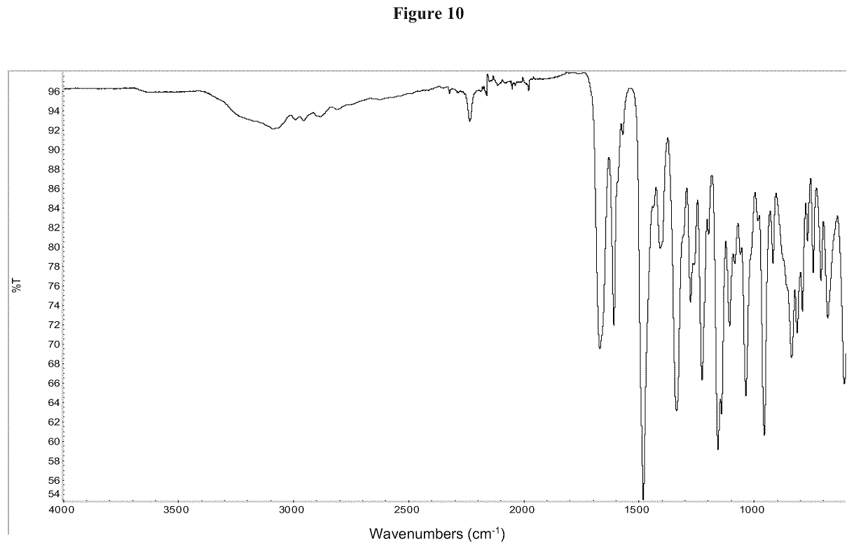

A certain embodiment relates to the amorpous form of the compound of formula (I) as described herein, characterized by the IR spectrum as shown in FIG. 10.

In one embodiment, the crystalline polymorphic Form A of compound of formula (I) is anhydrous, i.e. free of water bound in the crystal lattice, and non-hygroscopic (<0.2% water uptake according to European Pharmacopeia). In another embodiment, crystalline polymorphic Form A of the compound of formula (I) is substantially free of water and other solvents (in particular with ethanol <5000 ppm; H₂O <0.2% wt).

The invention thus also relates to a process for the preparation of a compound of formula (I),

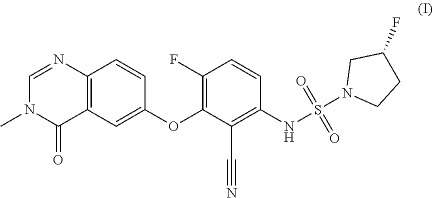 [SMILES: Cn1cnc2ccc(Oc3c(F)ccc(NS(=O)(=O)N4CC[C@@H](F)C4)c3C#N)cc2c1=O]

comprising one or two of the following steps:
(A) the reaction of a compound of formula (a1)

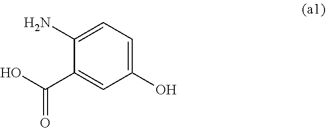 [SMILES: Nc1ccc(O)cc1C(=O)O]

with a compound of formula (a2)

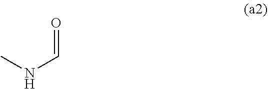

in presence of a suitable solvent, to arrive at a compound of formula (b1)

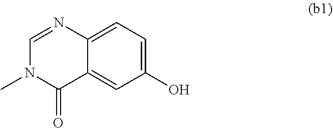

(B) the reaction of the compound of formula (b1) with a compound of formula (b2)

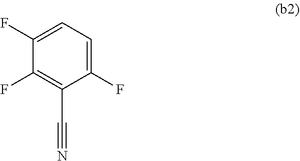 [SMILES: N#Cc1c(F)ccc(F)c1F]

in presence of a suitable base and a suitable solvent to arrive at a compound of formula (B2)

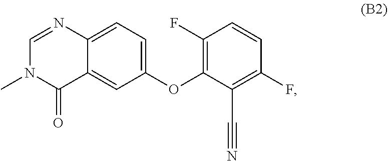 [SMILES: Cn1cnc2ccc(Oc3c(F)ccc(F)c3C#N)cc2c1=O]

wherein the base for step (B) is selected from potassium carbonate and sodium hydride.

In step (A) of the above process, the solvent can be for example 1,3-Dimethyl-2-imidazolidinone (DMI).

Conditions for step (A) can be between around 80° C. and around 200° C., particularly between around 100° C. and around 145° C., more particularly between around 120° C. and around 145° C. Conveniently, the reaction is kept at between around 30 minutes and around 36 hours, in particular between around 1 hour and around 30 hours, more particularly between around 16 hours and between around 26 hours.

Convenient conditions for step (A) can be between around 100° C. and around 145° C., using between around 2.0 equiv. and around10.0 equiv. of N-Methylformamide (NMP, a2) and between around 1.0 equiv. and around 10.0 equiv. of 1,3-Dimethyl-2-imidazolidinone (DMI).

Particularly convenient conditions for step (A) can be between around 120° C. and around 145° C., using between around 2.6 equiv. and around 7.2 equiv. of N-Methylformamide (NMP, a2) and between around 1.0 equiv. and around 3.0 equiv. of 1,3-Dimethyl-2-imidazolidinone (DMI).

In step (B) of the above process, the solvent can be for example DMF, water, acetone, acetonitrile or a mixture thereof, in particular acetone.

Conditions for step (B) can be between around 40° C. and around 120° C., particularly between around 60° C. and around 100° C., more particularly between around 70° C. and around 90° C. Conveniently, the reaction is kept at between around 30 minutes and around 36 hours, in particular between around 1 hour and around 30 hours, more particularly between around 16 hours and between around 26 hours.

Convenient conditions for step (B) can be between around 60° C. and around 100° C., using between around 5.0 and around 20 equiv. of a solvent selected from DMF, water, acetonitrile, acetone or a mixture thereof, and between around 1.15 equiv. and around 5.0 equiv. of a base selected from K₂CO₃ and sodium hydride.

Particularly convenient conditions for step (B) can be between around 60° C. and around 100° C., using beweent around 5.0 and around 10 equiv. of a solvent selected from DMF, water, acetonitrile, acetone or a mixture thereof, and between around 1.15 equiv. and around 2.0 equiv. of a base selected from K₂CO₃ and sodium hydride.

In one embodiment of the above process, the product of step (A) is isolated by an additional step (A-I), wherein a suitable solvent is added, the suspension is cooled and filtrated. In step (A-I), the solvent can be for instance water, ethyl acetate or a mixture thereof. Conveniently, the suspension is cooled to between around 0° C. to around 60° C., preferably to between around 10° C. to around 40° C., more preferentially to around 20° C.

The invention thus also relates to a process for the preparation of a compound of formula (I),

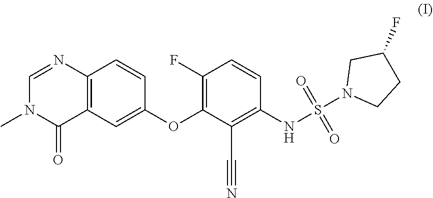 [SMILES: Cn1cnc2ccc(Oc3c(F)ccc(NS(=O)(=O)N4CC[C@@H](F)C4)c3C#N)cc2c1=O]

comprising one, two or three of the following steps:
(a) the reaction of a compound of formula (A1)

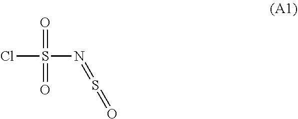

with a compound of formula (A2)

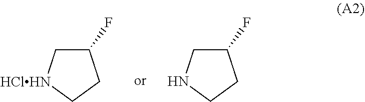

in presence of suitable base in presence of a suitable solvent, to arrive at a compound of formula (B1)

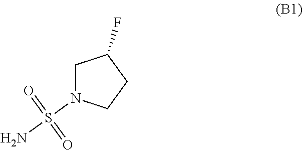

(b) the reaction of the compound of formula (B1) with a compound of formula (B2)

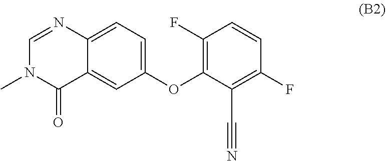 [SMILES: Cn1cnc2ccc(Oc3c(F)ccc(F)c3C#N)cc2c1=O]

in presence of a suitable base in presence of a suitable solvent to arrive at a compound of formula (I)

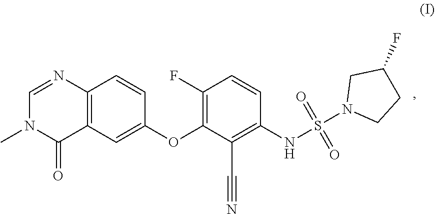 [SMILES: Cn1cnc2ccc(Oc3c(F)ccc(NS(=O)(=O)N4CC[C@@H](F)C4)c3C#N)cc2c1=O]

and
(c) wherein a suspension of the crude product of the compound of formula (I) in a suitable solvent and a suitable acid is purified by filtration to remove substantial amounts of dimer sulfate.

In one embodiment of the above process, a solvent selected from EtOH, water or a mixture is added to the reaction mixture at the end of step (b) to precipitate the title compound. The precipitate can then be filtrated and washed with EtOH and H₂O and dried in vacuo to afford the crude product (3R)-N-[2-cyano-4-fluoro-3-(3-methyl-4-oxo-quinazolin-6-yl)oxy-phenyl]-3-fluoro-pyrrolidine-1-sulfonamide, which is then subjected to step (c) of the above process.

In the above process, step (a) can conveniently be carried out in a solvent. The solvent can be for example tert-butanol, DCM or a mixture thereof, or ethyl acetate.

Step (a) can conveniently be carried out in presence of a base. The base can be for example DIPEA.

Convenient conditions for step (a) can be between 0° C. and around 30° C., particularly between around 1° C. and around 20° C., more particularly between around 1° C. and around 4° C. Conveniently the reaction is kept at the convenient temperature for between around 20 minutes and around 24 hrs, in particular between around 30 minutes and around 2 hrs.

Step (b) can conveniently be carried out in a solvent. The solvent can be for example DMF, acetonitrile, DMSO/water or 1,3-Dimethyl-2-imidazolidinone (DMI).

Step (b) can conveniently be carried out in presence of a base. The base can be for example cesium carbonate.

Conveniently in step (c) prior to the filtration at room temperature, the suspension is kept between around 50° C. and around 120° C., particularly between around 60° C. and around 110° C., more particularly between around 70° C. and around 100° C. Conveniently, the suspension is kept between around 30 minutes and around 10 hrs, more particularly between around 40 minutes and around 2 hrs. Convenient conditions are around 80° C. for around 1 h.

Step (c) can conveniently be carried out in a solvent. The solvent can be for example acetonitrile.

Step (c) can conveniently be carried out in presence of an acid. The base can be for example sulphuric acid.

A process for the preparation of crystalline polymorphic Form A of a compound of formula (I),

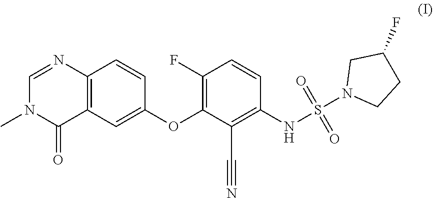 [SMILES: Cn1cnc2ccc(Oc3c(F)ccc(NS(=O)(=O)N4CC[C@@H](F)C4)c3C#N)cc2c1=O]

comprising at least one, two or three of the following steps:
(a) a filtrate as described herein comprising substantial amounts of the compound of formula (I) is concentrated under vacuum to provide a suspension;
(b) water and a suitable base are added to a suspension comprising the compound of formula (I) and after adjustment of the pH to 6.7±0.5 the reaction is stirred to afford a precipitate comprising the compound of formula (I);
(c) a precipitate comprising the compound of formula (I) is filtrated, then washed with a suitable solvent and dried in vacuo to afford crystallized (3R)-N-[2-cyano-4-fluoro-3-(3-methyl-4-oxo-quinazolin-6-yl)oxy-phenyl]-3-fluoro-pyrrolidine-1-sulfonamide;
(d) a suitable solvent and water are added to (3R)-N-[2-cyano-4-fluoro-3-(3-methyl-4-oxo-quinazolin-6-yl)oxy-phenyl]-3-fluoro-pyrrolidine-1-sulfonamide, the suspension is heated to between about 40° C. to about 80° C., in particular to about 60° C., and the resulting solution is filtered and washed with a suitable solvent;
(e) a solution comprising to (3R)-N-[2-cyano-4-fluoro-3-(3-methyl-4-oxo-quinazolin-6-yl)oxy-phenyl]-3-fluoro-pyrrolidine-1-sulfonamide in a suitable solvent is concentrated in vacuo;
(f) solvent exchange of a solution comprising (3R)-N-[2-cyano-4-fluoro-3-(3-methyl-4-oxo-quinazolin-6-yl)oxy-phenyl]-3-fluoro-pyrrolidine-1-sulfonamide by distillation and stirring the suspension to precipitate (3R)-N-[2-cyano-4-fluoro-3-(3-methyl-4-oxo-quinazolin-6-yl)oxy-phenyl]-3-fluoro-pyrrolidine-1-sulfonamide; and
(g) a suspension of (3R)-N-[2-cyano-4-fluoro-3-(3-methyl-4-oxo-quinazolin-6-yl)oxy-phenyl]-3-fluoro-pyrrolidine-1-sulfonamide in a suitable solvent is filtered and dried in vacuo to afford crystalline polymorphic Form A.
A convenient solvent for step (b) is for example water. A convenient base for step (b) is for example NaOH.
Convenient solvents for step (c) are for example acetonitrile and/or water.
Convenient solvents for step (d) are for example acetone and/or water.
Convenient solvents for step (e) are for example acetone and/or water.
Conveniently, in step (f) the solvent was exchanged from acetone/water to ethanol/water.
Conveniently, in step (g) the solvent is ethanol, water or a mixture thereof.

The invention also relates to a compound according to the invention when manufactured according to a process of the invention.

### Assay Procedures

### Materials

DMEM no-phenol red medium supplemented with L-glutamine was purchased from (Thermo Fisher Scientific). Fetal bovine serum (FBS) was purchased from VWR. Advanced ERK phospho-T202/Y204 kit—10,000 tests was purchased from Cisbio cat #64AERPEH. A375 and HCT116 cells were originally obtained from ATCC and banked by the Roche repository. 384-well microplates were purchased from Greiner Bio-One, 384-well, (With Lid, HiBase, Low volume cat 784-080).

### HTRF Assay for P-ERK Determination in A375 Cells or HCT116 Cells

A375 is a cellular cancer model expressing V600E mutated BRAF and HCT116 a cellular cancer model expressing WT BRAF. First generation BRAF inhibitors such as e.g. dabrafenib induce a paradox effect on tumour cells in that they inhibit the growth of V600E mutated BRAF cells (such as e.g. A375), while they activate growth in WT BRAF cells (such as e.g. HCT 116). ERK 1,2 phosphorylation (terminal member of the phosphorylation cascade of the MAPK pathway) is hereafter reported as main readout for the activation status of the MAPK pathway. Prior to the assay, A375 and HCT116 cell lines are maintained in DMEM no-phenol red medium supplemented with 10% fetal bovine serum (FBS). Following compound treatment, P-ERK levels are determined by measuring FRET fluorescence signal induced by selective binding of 2 antibodies provided in the mentioned kit (Cisbio cat #64AERPEH) on ERK protein when phosphorylated at Thr202/Tyr204. Briefly, 8000 cells/well in 12 μl media/well are plated in the 384-well plate and left overnight in the incubator (at 37° C. with 5% CO2-humidified atmosphere), the following day the plate is treated in duplicate with test compounds, dabrafenib and PLX8394 (the latter two as controls) at the following final drug concentrations: 10 μM-3 μM-1 μM-0.3 μM-0.1 μM- 0.03 μM-0.01 μM-0.003 μM-0.001 μM, all wells are subjected to DMSO normalization and drug incubation occurs for 1 hour. Then, 4 μl of a 4× lysis buffer supplied with the kit are added to the wells, the plate is then centrifuged for 30 second (300 rcf) and incubated on a plate shaker for 1 h at RT.

At the end of the incubation 4 μL/well of advanced P-ERK antibody solution (prepared according to manufacturer's instruction) followed by 4 μL/well of criptate P-ERK antibody solution (prepared according to manufacturer's instruction) (Cisbio cat #64AERPEH) are added to test wells.

In order to allow proper data normalization control wells without drug treatment reported are always included in each plate (according to manufacturer's instruction):
p-ERK HTRF well content of controls and experimental (μl):

| neg ctrl | pos ctrl | neut ctrl | cpd | blank | |
|---|---|---|---|---|---|
| — | — | 12 | 12 | 12 | Cells |
| 12 | — | — | — | — | Media |
| — | — | — | <0.05 | — | Cpd |
| — | 16 | — | — | — | control lysate (ready-to-use) |
| 4 | — | 4 | 4 | 4 | 4x lysis buffer |
| 4 | 4 | 4 | 4 | — | Advanced p-ERK |
| | | | | | antibody solution |
| — | — | — | — | 4 | Advanced p-ERK1/2 |
| | | | | | Cryptate antibody |
| | | | | | solut. |
| 20 | 20 | 20 | 20 | 20 | Total volume in Well |

The plate is then centrifuged at 300 rcf for 30 second, sealed to prevent evaporation and incubated overnight in the dark at room temperature.

The plate is then analyzed and fluorescence emission value collected through a Pherastast FSX (BMG Labtech) apparatus at 665 and 620 nM.

The obtained fluorescence values are processed according to the formula Ratio=Signal(620 nm)/Signal(625 nm)*10000 then the average of the ratio on the blank is subtracted to all values.

Data are normalized in the case of A375 cells (BRAF inhibition) considering the average of the ratio (blank subtracted) derived by DMSO only treated cells as 100% and by considering the average of the ratio (blank subtracted) derived by 10 μM dabrafenib treated cells as 0%. Mean of the normalized points are fitted with sigmoidal curve and IC50 determined. The results are shown in Table 1.

Data are normalized in the case of HCT116 cells (BRAF activation,) considering the average of the ratio (blank subtracted) derived by DMSO only treated cells as 0% and by considering the average of the ratio (blank subtracted) derived by dabrafenib treated cells at the concentration which provides the highest signal as 100%. Individual points are fitted with either sigmoidal or bell shape curves, and the percentage of activation compared to maximum dabrafenib-mediated activation is determined. The EC50 is the concentration at which activation equal to 50% of the maximum achieved by dabrafenib is obtained. The results are shown in Table 2.

In case the activation does not reach 50% of the maximum achieved by dabrafenib, then the EC50 calculation is not applicable.

The Percentage of Maximum paradox inducing effect from dabrafenib is determined by evaluating the percentage at which the test compound induce its maximum P-ERK signal as percentage of the highest signal produced by dabrafenib within the dose range tested.

WO2012/118492 discloses references compounds AR-25 as example 25, AR-30 as example 30 and AR-31 as example 31.

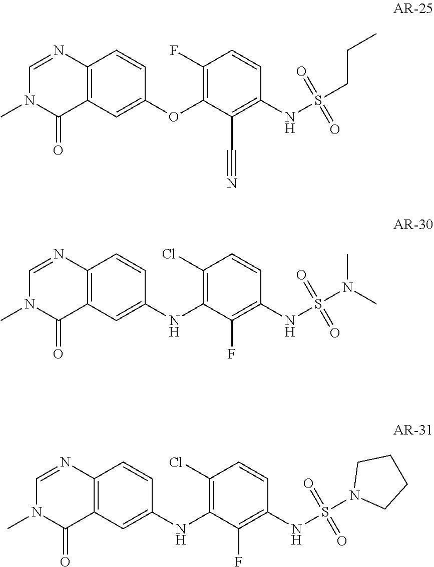

TABLE 1
Example 1 ((3R)-N-[2-cyano-4-fluoro-3-(3-methyl-4-oxo-quinazolin-
6-yl)oxy-phenyl]-3-fluoro-pyrrolidine-1-sulfonamide) has
high affinity for RAF kinases and high selectivity over C-
terminal Src kinase (CSK) and lymphocyte-specific tyrosine
protein kinase (LCK), when compared to AR-30 and AR-31.

Kd (μM)

| | | BRAF | | | |
|---|---|---|---|---|---|
| Ex. | BRAF | V600E | CRAF | CSK | LCK |
| 1 | 0.0006 | 0.0012 | 0.0017 | 23.3 | 40 |
| AR-25 | 0.0001 | 0.0002 | 0.0003 | >40 | >40 |
| AR-30 | 0.1740 | 0.5040 | 0.8220 | 8.007 | 10.352 |
| AR-31 | 0.0459 | 0.1190 | 0.1903 | 1.208 | 11.975 |

TABLE 2
Example 1 ((3R)-N-[2-cyano-4-fluoro-3-(3-methyl-4-
oxo-quinazolin-6-yl)oxy-phenyl]-3-fluoro-pyrrolidine-
1-sulfonamide) is breaking the paradoxical RAF activation
in HCT-116 cancer cells expressing WT BRAF. When compared
with dabrafenib or with AR-25 the maximum paradox inducing
effect is reduced to less than 50%.

| | | p-ERK EC50 (nM) conc. (nM) | |
|---|---|---|---|
| | | at which the compound induces | |
| | | P-ERK activation of 50% of | |
| | pERK | that induced by dabrafenib | Percentage of |
| | IC₅₀ | (Positive control | Maximum paradox |
| | (nM) | paradox inducer) | inducing effect |
| Ex. | A375 | HCT-116 | from dabrafenib |
| 1 | 6.9 | not applicable | 43.65% |
| AR-25 | 1.1 | 9.6 | 103% |
| AR-30 | 406 | >1000 | 59% |
| AR-31 | 311 | >1000 | 51.2% |

### Pharmaceutical Compositions

The compound of formula (I) can be used as therapeutically active substance, e.g. in the form of a pharmaceutical composition. The pharmaceutical composition can be administered orally, e.g. in the form of tablets, coated tablets, dragées, hard and soft gelatin capsules, solutions, emulsions or suspensions. The administration can, however, also be effected rectally, e.g. in the form of suppositories, or parenterally, e.g. in the form of injection solutions.

The compound of formula (I) can be processed with a pharmaceutically inert, inorganic or organic carriers for the production of a pharmaceutical composition. Lactose, corn starch or derivatives thereof, talc, stearic acids or its salts and the like can be used, for example, as such carriers for tablets, coated tablets, dragées and hard gelatin capsules. Suitable carriers for soft gelatin capsules are, for example, vegetable oils, waxes, fats, semi-solid and liquid polyols and the like. Depending on the nature of the active substance no carriers are however usually required in the case of soft gelatin capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical composition can, moreover, contain pharmaceutically acceptable auxiliary substances such as preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

Pharmaceutical compositions comprising a compound of formula (I) alone or in combination, can be prepared for storage by mixing the active ingredient having the desired degree of purity with optional pharmaceutically acceptable carriers, excipients or stabilizers (Remington's Pharmaceutical Sciences 16th edition, Osol, A. (ed.) (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Medicaments containing the compound of formula (I) and a therapeutically inert carrier are also provided by the present invention, as is a process for their production, which comprises bringing one or more compounds of formula (I) and/or pharmaceutically acceptable solvates thereof and, if desired, one or more other therapeutically valuable substances into a galenical administration form together with one or more therapeutically inert carriers.

Pharmaceutical compositions of a BRAF inhibitor include those suitable for oral, nasal, topical (including buccal and sublingual), rectal, vaginal and/or parenteral administration.

The dosage can vary within wide limits and will, of course, have to be adjusted to the individual requirements in each particular case. In the case of oral administration the dosage for adults can vary from about 0.01 mg to about 1000 mg per day of a compound of general formula (I) or of the corresponding amount of a pharmaceutically acceptable solvate thereof. The daily dosage may be administered as single dose or in divided doses and, in addition, the upper limit can also be exceeded when this is found to be indicated.

The following examples illustrate the present invention without limiting it, but serve merely as representative thereof. The pharmaceutical preparations conveniently contain about 1-500 mg, particularly 1-100 mg, of a compound of formula (I). Examples of compositions according to the invention are:

### Example A

Tablets of the following composition are manufactured in the usual manner:

TABLE 3
possible tablet composition

mg/tablet

| ingredient | 5 | 25 | 100 | 500 |
|---|---|---|---|---|
| Compound of formula (I) | 5 | 25 | 100 | 500 |
| Lactose Anhydrous DTG | 125 | 105 | 30 | 150 |
| Sta-Rx 1500 | 6 | 6 | 6 | 60 |
| Microcrystalline Cellulose | 30 | 30 | 30 | 450 |
| Magnesium Stearate | 1 | 1 | 1 | 1 |
| Total | 167 | 167 | 167 | 831 |

### Manufacturing Procedure

1. Mix ingredients 1, 2, 3 and 4 and granulate with purified water.
2. Dry the granules at 50° C.
3. Pass the granules through suitable milling equipment.
4. Add ingredient 5 and mix for three minutes; compress on a suitable press.

### Example B-1

Capsules of the following composition are manufactured:

TABLE 4
possible capsule ingredient composition

mg/capsule

| ingredient | 5 | 25 | 100 | 500 |
|---|---|---|---|---|
| Compound of formula (I) | 5 | 25 | 100 | 500 |
| Hydrous Lactose | 159 | 123 | 148 | — |
| Corn Starch | 25 | 35 | 40 | 70 |
| Talk | 10 | 15 | 10 | 25 |
| Magnesium Stearate | 1 | 2 | 2 | 5 |
| Total | 200 | 200 | 300 | 600 |

### Manufacturing Procedure

1. Mix ingredients 1, 2 and 3 in a suitable mixer for 30 minutes.
2. Add ingredients 4 and 5 and mix for 3 minutes.
3. Fill into a suitable capsule.

The compound of formula (I), lactose and corn starch are firstly mixed in a mixer and then in a comminuting machine. The mixture is returned to the mixer; the talc is added thereto and mixed thoroughly. The mixture is filled by machine into suitable capsules, e.g. hard gelatin capsules.

### Example B-2

Soft Gelatin Capsules of the following composition are manufactured:

TABLE 5
possible soft gelatin capsule ingredient composition

| ingredient | mg/capsule |
|---|---|
| Compound of formula (I) | 5 |
| Yellow wax | 8 |
| Hydrogenated Soya bean oil | 8 |
| Partially hydrogenated plant oils | 34 |
| Soya bean oil | 110 |
| Total | 165 |

TABLE 6
possible soft gelatin capsule composition

| ingredient | mg/capsule |
|---|---|
| Gelatin | 75 |
| Glycerol 85% | 32 |
| Karion 83 | 8 (dry matter) |
| Titan dioxide | 0.4 |
| Iron oxide yellow | 1.1 |
| Total | 116.5 |

### Manufacturing Procedure

The compound of formula (I) is dissolved in a warm melting of the other ingredients and the mixture is filled into soft gelatin capsules of appropriate size. The filled soft gelatin capsules are treated according to the usual procedures.

### Example C

Suppositories of the following composition are manufactured:

TABLE 7
possible suppository composition

| ingredient | mg/supp. |
|---|---|
| Compound of formula (I) | 15 |
| Suppository mass | 1285 |
| Total | 1300 |

### Manufacturing Procedure

The suppository mass is melted in a glass or steel vessel, mixed thoroughly and cooled to 45° C. Thereupon, the finely powdered compound of formula (I) is added thereto and stirred until it has dispersed completely. The mixture is poured into suppository moulds of suitable size, left to cool; the suppositories are then removed from the moulds and packed individually in wax paper or metal foil.

### Example D

Injection solutions of the following composition are manufactured:

TABLE 8
possible injection solution composition

| ingredient | mg/injection solution. |
|---|---|
| Compound of formula (I) | 3 |
| Polyethylene Glycol 400 | 150 |
| acetic acid | q.s. ad pH 5.0 |
| water for injection solutions | ad 1.0 ml |

### Manufacturing Procedure

The compound of formula (I) is dissolved in a mixture of Polyethylene Glycol 400 and water for injection (part). The pH is adjusted to 5.0 by acetic acid. The volume is adjusted to 1.0 ml by addition of the residual amount of water. The solution is filtered, filled into vials using an appropriate overage and sterilized.

### Example E

Sachets of the following composition are manufactured:

TABLE 9
possible sachet composition

| ingredient | mg/sachet |
|---|---|
| Compound of formula (I) | 50 |
| Lactose, fine powder | 1015 |
| Microcrystalline cellulose (AVICEL PH 102) | 1400 |
| Sodium carboxymethyl cellulose | 14 |
| Polyvinylpyrrolidon K 30 | 10 |
| Magnesium stearate | 10 |
| Flavoring additives | 1 |
| Total | 2500 |

### Manufacturing Procedure

The compound of formula (I) is mixed with lactose, microcrystalline cellulose and sodium carboxymethyl cellulose and granulated with a mixture of polyvinylpyrrolidone in water. The granulate is mixed with magnesium stearate and the flavoring additives and filled into sachets.

### EXAMPLES

The following examples are provided for illustration of the invention. They should not be considered as limiting the scope of the invention, but merely as being representative thereof.

### Abbreviations

ATR=attenuated total reflection; DCM=dichloromethane; DIPEA=N,N-diisopropylethylamine; DMF=dimethylformamide; DMSO dimethyl sulfoxide; DSC=differential scanning calorimetry; DVS=dynamic vapour sorption; ESI=electrospray ionization; EtOAc=ethyl acetate; FT=fourier transform; FTIR=fourier-transform infrared; IR=infrared; LC-MS/MS=liquid chromatography-MS/MS; MeOH=methanol; MS=mass spectrometry; RH=relative humidity; rt=room temperature; SFC=supercritical fluid chromatography; TGA=thermogravimetry.

### High Resolution X-Ray Powder Diffraction

High resolution X-ray powder diffraction (XRPD) patterns were recorded either in transmission geometry. X-ray diffraction patterns were recorded on a STOE STADI P diffractometer with CuKa1 radiation (1.5406 Å) and a Mythen position sensitive detector. The samples (approximately 10 to 50 mg) were prepared between thin polymer films and were usually analyzed without further processing (e.g., grinding or sieving) of the substance.

For polymorphic Form A the following peaks have been found by XRPD (expressed in values of degrees 2-theta) at approximately: 5.06; 7.90; 8.92; 9.88; 10.22; 11.28; 11.58; 12.16; 12.66; 13.16; 13.64; 14.66; 14.84; 15.38; 15.66; 15.86; 16.24; 16.54; 17.18; 17.50; 17.72; 18.06; 18.58; 18.86; 18.98; 19.40; 19.64; 20.54; 20.72; 21.18; 22.26; 22.56; 23 .; 23.30; 23.90; 24.08; 24.44; 25.16; 25.46; 25.78; 26.04; 26.16; 26.40; 26.66; 27.28; 27.82; 28.26; 28.40; 28.76; 28.92; 29.36; 29.58; 29.96; 30.28.

### Differential Scanning Calorimetry (DSC)

DSC curves were recorded using a Mettler-Toledo™ differential scanning calorimeter DSC2. System suitability tests were performed with Indium as reference substance and calibrations were carried out using Indium, Benzoic acid, Biphenyl and Zinc as reference substances.

For the measurements, approximately 2 to 6 mg of sample were placed in aluminum pans, accurately weighed and hermetically closed with perforation lids. Prior to measurement, the lids were pierced resulting in approx. 0.5 mm pin holes. The samples were then heated under a flow of nitrogen of about 100 mL/min using heating rates of typically 1 to 20, usually 10 K/min to a maximum temperature of typically 180° C. to 350° C. depending on decomposition temperature.

### Thermogravimetry (TGA)

Thermogravimetric analyses (TGA) were performed on a Mettler-Toledo™ thermogravimetric analyzer (TGA/DSC1 or TGA/DSC3+). System suitability tests were performed with Hydranal as reference substance and calibrations using Aluminum and Indium as reference substances.

For the thermogravimetric analyses, approx. 5 to 15 mg of sample were placed in aluminum pans, accurately weighed and hermetically closed with perforation lids. Prior to measurement, the lids were automatically pierced resulting in approx. 0.5 mm pin holes. The samples were then heated under a flow of nitrogen of about 50 mL/min using a heating rate of 5 K/min to a maximum temperature of typically 350° C.

### Moisture Sorption/Desorption

Moisture sorption/desorption data was collected on a DVS Advantage, a DVS Adventure, or a DVS Intrinsic (SMS Surface Measurements Systems) moisture balance system. The sorption/desorption isotherms were measured stepwise in a range from 0%-RH to 90%-RH at typically 25° C. A weight change of typically <0.001%/min was chosen as criterion to switch to the next level of relative humidity (with a maximum equilibration time of typically 24 hours, if the weight change criterion was not met). The data were corrected for the initial moisture content of the samples by taking the weight after drying of the samples at 0%-RH as zero point.

The hygroscopicity of a given substance was characterized (by close analogy with the European Pharmacopoeia) by the increase in mass when the relative humidity was raised from 0%-RH to 90%-RH:

| | | |
|---|---|---|
| non-hygroscopic: | weight increase | Dm < 0.2% |
| slightly hygroscopic: | weight increase | 0.2% ≤ Dm < 2.0% |
| hygroscopic: | weight increase | 2.0% ≤ Dm < 15.0% |
| very hygroscopic: | weight increase | Dm ≥ 15.0% |

| | |
|---|---|
| deliquescent: | sufficient liquid is adsorbed to form a liquid |

### European Pharmacopoeia—8^{th} Edition (2014), Chapter 5.11.

### IR Spectroscopy

The ATR FTIR spectra were recorded without any sample preparation using a ThermoNicolet iS5 FTIR spectrometer with ATR accessory. The spectral range was between 4000 cm⁻¹ and 650 cm⁻¹, resolution 2 cm⁻¹, and at least 50 co-added scans were collected. Happ-Genzel apodization was applied. Using ATR FTIR will cause the relative intensities of infrared bands to differ from those seen in a transmission FTIR spectrum using KBr disc or nujol mull sample preparations. Due to the nature of ATR FTIR, the bands at lower wavenumber are more intense than those at higher wavenumber.

Peakpicking was performed using Thermo Scientific Omnic 8.3 software using the automated ‘Find Peaks’ function. The ‘threshold’ and ‘sensitivity’ were manually adjusted to get a representative number of peaks.

TABLE 11
list of peaks identified by infrared
spectroscopy of polymorphic Form A.

3139
2874
2231
1656
1607
1590
1569
1493
1481
1434
1413
1397
1348
1326
1279
1264
1227
1201
1159
1141
1106
1057
1043
1011
961
931
892
863
838
823
815
794
770
745
708
689
662

TABLE 12
list of peaks identified by infrared
spectroscopy of amorphous form.

3092
2235
1669
1608
1570
1481
1407
1337
1275
1225
1197
1156
1141
1106
1084
1035
956
918
836
812
789
767
742
708
679

### Raman Spectroscopy

The FT-Raman spectra were recorded without any sample preparation in the spectral range of 4000-50 cm⁻¹ with a Bruker MultiRam FT-Raman spectrometer, equipped with a NdYAG 1064 nm laser and a liquid nitrogen cooled Germanium detector. The laser power at the sample was about 300 mW, 2 cm⁻¹ resolution was used, and 2048 scans were co-added. The Blackman-Harris 4-term apodization function was used. About 5 mg of sample (powder in a glass vial) were needed. Peakpicking was performed using Thermo Scientific Omnic 8.3 software using the automated ‘Find Peaks’ function. The ‘threshold’ and ‘sensitivity’ were manually adjusted to get a representative number of peaks.

TABLE 13
list of peaks identified by Raman
spectroscopy of polymorphic Form A.

3083
3061
2984
2966
2233
1660
1607
1570
1483
1431
1398
1350
1311
1279
1229
1213
1151
1058
1012
962
921
786
770
747
691
617
572
534
493
439
426
415
323
302
175
146
68

TABLE 14
list of peaks identified by Raman spectroscopy of amorphous form.

3069
2995
2958
2236
1676
1614
1569
1484
1429
1398
1344
1308
1276
1228
1159
1060
1010
960
919
783
766
744
709
684
613
575
543
482
436
415
340
302
61

### Synthesis

### (3R)-N-[2-cyano-4-fluoro-3-(3-methyl-4-oxo-quinazolin-6-yl)oxy-phenyl]-3-fluoro-pyrrolidine-1-sulfonamide

### Step 1: 6-hydroxy-3-methyl-quinazolin-4-one

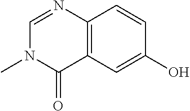

2-Amino-5-hydroxybenzoic acid (10 g, 65.3 mmol, Eq: 1.0) and N-methylformamide (30 g, 29.9 mL, 503 mmol, Eq: 7.7) were heated at 145° C. for 21 h 45 min, then cooled to rt. The reaction mixture was diluted with 50 mL H₂O and stirred at rt for 20 min. The resulting precipitate was collected by filtration. The light brown solid was washed 3× with 20 mL water. The solid was taken up in toluene and evaporated to dryness (3×). The solid was dried in vacuo at 40° C. overnight under high vacuum to give the title compound as a light brown solid (10.3 g, 89% yield). MS (ESI) m/z: 177.1 [M+H]⁺.

### Step 2: 3,6-difluoro-2-(3-methyl-4-oxo-quinazolin-6-yl)oxy-benzonitrile

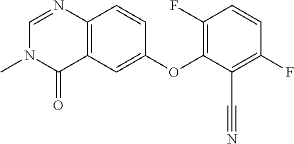 [SMILES: Cn1cnc2ccc(Oc3c(F)ccc(F)c3C#N)cc2c1=O]

Cesium carbonate (3.22 g, 9.79 mmol, Eq: 1.15) was added at rt to a solution of 6-hydroxy-3-methylquinazolin-4-one (1500 mg, 8.51 mmol, Eq: 1.0) in N,N-dimethylformamide (35 mL). The mixture was stirred for 30 min at rt then 2,3,6-trifluorobenzonitrile (1.47 g, 1.08 ml, 9.37 mmol, Eq: 1.1) was added. After 1 h, the reaction was cooled on ice and diluted with water (120 mL). The resultant solid was collected by filtration, washed with iced water (100 mL) and heptane (100 mL) and suction-dried. The solid was taken up in toluene and evaporated to dryness (3×) then dried overnight in vacuo to give the title compound as a light brown solid (2.58 g, 97% yield). MS (ESI) m/z: 314.1 [M+H]+.

### Step 3: (3R)-3-fluoropyrrolidine-1-sulfonamide

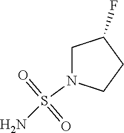

(R)-3-Fluoropyrrolidine hydrochloride (1.8 g, 14.3 mmol, Eq: 1.2) was added to a solution of sulfuric diamide (1.148 g, 11.9 mmol, Eq: 1.0) and triethylamine (2.42 g, 3.33 mL, 23.9 mmol, Eq: 2) in dioxane (10 mL). The reaction was stirred in a sealed tube at 115° C. for 15.5 h then cooled to rt and concentrated in vacuo. The residue was diluted with DCM, evaporated with silica gel to dryness and transferred to a column. Purification by flash chromatography (40 g silica, 80% EtOAc) gave the title compound as a white crystalline solid (1.82 g, 91% yield). MS (ESI) m/z: 169.1 [M+H]⁺.

### Step 4: (3R)-N-[2-cyano-4-fluoro-3-(3-methyl-4-oxo-quinazolin-6-yl)oxy-phenyl]-3-fluoro-pyrrolidine-1-sulfonamide

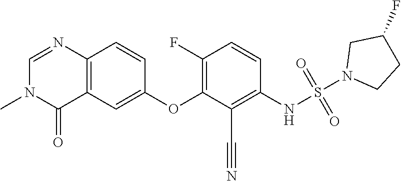 [SMILES: Cn1cnc2ccc(Oc3c(F)ccc(NS(=O)(=O)N4CC[C@@H](F)C4)c3C#N)cc2c1=O]

(R)-3-Fluoropyrrolidine-1-sulfonamide (1.26 g, 7.51 mmol, Eq: 2.1) and cesium carbonate (2.56 g, 7.87 mmol, Eq: 2.2) were suspended in dry DMF (10.2 ml) under an argon atmosphere. The reaction was stirred at 50° C. for 30 min. The reaction mixture was cooled to rt and a solution of 3,6-difluoro-2-((3-methyl-4-oxo-3,4-dihydroquinazolin-6-yl)oxy)benzonitrile (1.12 g, 3.58 mmol, Eq: 1.0) in DMF (25.5 ml) was added. The reaction mixture was stirred at 100° C. for 15 h, then concentrated in vacuo. The residue was taken up in sat. aq. NH₄Cl (100 mL) and EtOAc (100 mL). The phases were separated, and the aqueous layer was extracted further with 2×100 mL EtOAc. The combined organic layers were washed with water (200 mL) and brine (200 mL), dried (Na₂SO₄), filtered and concentrated in vacuo. The water layer was back-extracted with EtOAc (3×100 mL). The combined organic extracts were washed with brine (200 mL), dried (Na₂SO₄), filtered and concentrated in vacuo. The residue was diluted with DCM and MeOH, and concentrated onto silica. Purification by flash chromatography (120 g, 0.5-2% MeOH/DCM) gave an off-white solid which was triturated with 1:1 heptane/DCM (20 mL) with sonication, then dried in vacuo to give the title compound (Example 1) as a colourless solid (1.087 g, 66% yield). MS (ESI) m/z: 426.2 [M+H]⁺. Chiral SFC: RT=4.594 min [Chiralpak IC column, 4.6×250 mm, 5 μm particle size (Daicel); gradient of 20-40% MeOH containing 0.2% NHEt₂ over 8 min; flow: 2.5 mL/min; 140 bar backpressure].

### (3R)-N-[2-cyano-4-fluoro-3-(3-methyl-4-oxo-quinazolin-6-yl)oxy-phenyl]-3-fluoro-pyrrolidine-1-sulfonamide—Alternative Synthesis

### Step 1: 6-hydroxy-3-methyl-quinazolin-4-one

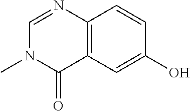

2-amino-5-hydroxybenzoic acid (1 eq.) was suspended in N-methylformamide (2.6 eq.) and 1,3-Dimethyl-2-imidazolidinone (2.96 eq). The suspension was heated at 140° C. for 21 hours and then cooled at 90° C. for 1 hour. Water (3.1 vol) was added slowly and the suspension was cooled to 20° C. in 2 h. Additional water was added (0.15 vol) then the solid was allowed to settle at the bottom of the reactor for 1.5 h. The supernatent was removed by canula, then water (2.6 vol) was added and the suspension was stirred. The solid was allowed to settle at the bottom of the reactor and the supernatent was then removed by canula. This step was repeated another 5 times. Finally water (2.6 vol) was added to the residual suspension, stirred and filtrated. The filter cake was washed with water (0.7 vol). The product was dried in vacuo to afford the the title compound as a brown crystalline solid (73% yield).

### Step 2: 3,6-difluoro-2-(3-methyl-4-oxo-quinazolin-6-yl)oxy-benzonitrile

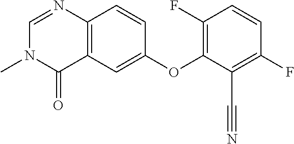 [SMILES: Cn1cnc2ccc(Oc3c(F)ccc(F)c3C#N)cc2c1=O]

6-hydroxy-3-methyl-quinazolin-4-one (1 eq), potassium carbonate (1.15 eq), 2,3,6-trifluorobenzonitrile (1.1 eq) and acetone (5.2 vol) were added to a nitrogen flushed reactor. The content was heated at 80° C. for 16 hours. The mixture was cooled to 20° C. and water (10 vol) was added. The slurry was stirred for 0.5 hour and filtered. The filter cake was washed with water (2.4 vol). The product was dried in vacuo to afford the the title compound as a white crystalline solid (96.34%).

### Step 3: (3R)-3-fluoropyrrolidine-1-sulfonamide

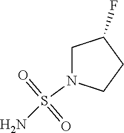

1.13 eq t-BuOH and DCM (6.8 vol) were added to a nitrogen flushed reactor and cooled to 0° C. 1.08 eq. chlorosulfonyl isocyanate was added while keeping the reaction temperature at −2° C. to 4° C. The addition vessel was washed with dichloromethane (0.34 vol). and the mixture was stirred for 30 min. 1 eq (R)-(−)-3-Fluoropyrrolidin HCl was added followed by 2.41 eq of diisopropylethylamine keeping the reaction temperature at 1° C. to 4° C. The solution was stirred for 1 h at 0° C. and warmed to 25° C. The organic layer was extracted with water (3.4 vol) and hydrochloric acid (0.33 equiv), then with water (1.7 vol). The solvent was distilled and the residual solid was dissolved in 15 L 1-propanol (2.6 vol). Then a prepared solution of HCl (1.5 equiv.) in 1-propanol (6.8 vol) was added in 30 minutes at 50° C. The solution was further stirred for 1 h. The solvent was then exchanged by distillation with toluene (total 16.1 vol) while keeping the volume constant and stirred overnight at rt. The suspension was filtered and washed with toluene (2 vol) and dried in vacuo to afford the the title compound as an off-white crystalline solid (88%)

### Step 4: (3R)-N-[2-cyano-4-fluoro-3-(3-methyl-4-oxo-quinazolin-6-yl)oxy-phenyl]-3-fluoro-pyrrolidine-1-sulfonamide

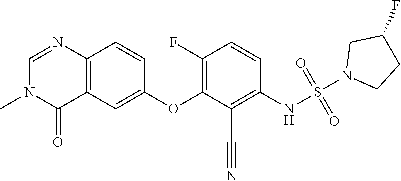 [SMILES: Cn1cnc2ccc(Oc3c(F)ccc(NS(=O)(=O)N4CC[C@@H](F)C4)c3C#N)cc2c1=O]

(R)-3-Fluoropyrrolidine-1-sulfonamide (1.06 eq.) in DMF (5.39 eq.) was slowly added to a solution of cesium carbonate (2.14 eq.) and 3,6-difluoro-2-((3-methyl-4-oxo-3,4-dihydroquinazolin-6-yl)oxy)benzonitrile (1.0 eq.) suspended in dry DMF (12.59 eq.) at 90° C. under an argon atmosphere. The reaction was stirred at around 90° C. for about 16 hrs. The reaction mixture was maintained at around 70° C. and acetic acid (4.06 eq.) was added over 20 minutes. The reaction mixture was cooled to rt and EtOH was added (initially 23.84 eq. at once, then additionally 26.23 eq. over 1 h) to precipitate the title compound. The precipitate was filtrated, then washed with EtOH and H₂O and dried in vacuo to afford the crude product (3R)-N-[2-cyano-4-fluoro-3-(3-methyl-4-oxo-quinazolin-6-yl)oxy-phenyl]-3-fluoro-pyrrolidine-1-sulfonamide. The crude product (1.0 eq.) was further purified by addition of acetonitrile (70.57 eq.) and sulfuric acid (1.34 eq.) at rt. The suspension was heated to 80° C. and stirred for 1 h, then the suspension was cooled at RT. The suspension was filtrated to remove dimer sulfate and the filter washed with acetonitrile (28.33 eq.) and H₂O and sodium hydroxide were added to the filtrate during 45 minutes and stirred over night at rt.

Crystallization steps: The solution was concentrated under vaccum to provide a suspension. Water was added to the suspension during 45 minutes and stirred for 1 h. The pH of the suspension was corrected to pH 6.7 by addition of water and sodium hydroxide (0.07 eq.) during 15 min. The suspension was stirred overnight at rt.

The precipitate was filtrated, then washed with acetonitrile (0.003 eq.) and H₂O and dried in vacuo to afford the crystallized (3R)-N-[2-cyano-4-fluoro-3-(3-methyl-4-oxo-quinazolin-6-yl)oxy-phenyl]-3-fluoro-pyrrolidine-1-sulfonamide.

To (3R)-N-[2-cyano-4-fluoro-3-(3-methyl-4-oxo-quinazolin-6-yl)oxy-phenyl]-3-fluoro-pyrrolidine-1-sulfonamide was added acetone (50.95 eq.) and water, and the suspension was heated to 60° C. The resulting solution was filtered and the filter was washed with acetone and water. The solution was concentrated at 50° C. under vaccuum then stirred overnight. The acetone was then exchanged with ethanol (106.79 eq.) by distillation and keeping the volume constant. The resulting suspension was stirred at rt overnight.

The precipitate was filtrated, then washed with ethanol (16.02 eq.) and dried in vacuo to afford the polymorphic Form A of the title compound as a white crystalline solid (94.24% yield).

### Procedure to Obtain the Compound of Formula (I) in Amorphous Form:

6.31 g of (3R)-N-[2-cyano-4-fluoro-3-(3-methyl-4-oxo-quinazolin-6-yl)oxy-phenyl]-3-fluoro-pyrrolidine-1-sulfonamide (Example 1) were dissolved in 119.9 g Acetone/Water (90%/10%).

The solution was spraydried with the following settings: Büchi Mini Spray Dryer B-290; Aspirator 100%; Nitrogen 5 bar; Inlet Temp. 160° C.; Outlet without solvent 89° C.; Pump: 7.65 g/min; Outlet with Acetone/Water: 81° C.; Outlet with 5% BRAF inhibitor: decreases to 72° C.; Duration of Spraydrying: 16.5min;

Yield=3.13 g of amorphous (3R)-N-[2-cyano-4-fluoro-3-(3-methyl-4-oxo-quinazolin-6-yl)oxy-phenyl]-3-fluoro-pyrrolidine-1-sulfonamide

### Cell Line and Culture Conditions

Cell lines were obtained from ATCC, maintained in humidified incubators at 5% CO2 in standard conditions and passaged twice a week. Culture conditions are reported below:

| Cell line | catalog#/origin | culture condition |
|---|---|---|
| A375 | CRL-1619 | Dulbecco's Modified Eagle's |
| | | Medium, high glucose, GlutaMAX |
| | | (DMEM GIBCO #10566016), 10% |
| | | Fetal Bovine Serum (FBS, GIBCO |
| | | #10270-106) |
| A375-NRAS | CRL-1619IG-2 | Dulbecco's Modified Eagle's |
| | | Medium, high glucose, GlutaMAX |
| | | (DMEM GIBCO #10566016), 10% |
| | | Fetal Bovine Serum (FBS, GIBCO |
| | | #10270-106) |
| HCT116 | ATCC # CCL-247 | Dulbecco's Modified Eagle's |
| | | Medium, high glucose, GlutaMAX |
| | | (DMEM GIBCO #10566016), 10% |
| | | Fetal Bovine Serum (FBS, GIBCO |
| | | #10270-106) |

Although the foregoing invention has been described in some detail by way of illustration and example for purposes of clarity of understanding, the descriptions and examples should not be construed as limiting the scope of the invention. The disclosures of all patent, patent applications and scientific literature cited herein are expressly incorporated in their entirety by reference.

1. A solid form of a compound of formula (I): 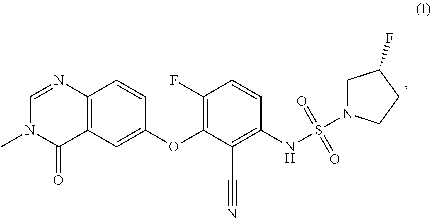 [SMILES: Cn1cnc2ccc(Oc3c(F)ccc(NS(=O)(=O)N4CC[C@@H](F)C4)c3C#N)cc2c1=O]
wherein the solid form is crystalline polymorphic Form A characterized by a X-ray powder diffraction pattern comprising a peak at an angle of diffraction at about 10.22 degrees 2-theta and at least one additional peak expressed in values of degrees 2-theta at approximately 7.90, 8.92, 11.58, 12.16, 12.66, 14.66, 17.50, 18.06, 19.64 or 20.54.

2. The solid form according to claim 1, characterized by a X-ray powder diffraction pattern comprising a peak at an angle of diffraction at about 10.22 degrees 2-theta and at about 18.06 degrees 2-theta.

3. The solid form according to claim 1, characterized by a X-ray powder diffraction pattern comprising a peak at an angle of diffraction at about 10.22 degrees 2-theta and at about 20.54 degrees 2-theta.

4. The solid form according to claim 1, characterized by a X-ray powder diffraction pattern comprising at least three of the peaks at an angle of diffraction at about 7.90, 8.92, 10.22, 11.58, 12.16, 12.66, 14.66, 17.50, 18.06, 19.64 and 20.54 degrees 2-theta.

5. The solid form according to claim 1, characterized by a X-ray powder diffraction pattern comprising the peaks at an angle of diffraction at about 7.90, 8.92, 10.22, 11.58, 12.16, 12.66, 14.66, 17.50, 18.06, 19.64 and 20.54 degrees 2-theta.

6. The solid form characterized by a X-Ray powder diffraction pattern according to claim 1, which is further comprising at least one additional peak expressed in values of degrees 2-theta at approximately 5.06, 9.88, 11.28, 13.16, 13.64, 14.84, 15.38, 15.66, 15.86, 16.24, 16.54, 17.18, 18.58, 18.98, 19.40, 20.72, 21.18, 22.26, 23.00, 23.30, 23.90, 24.08 or 24.44.

7. The solid form according to claim 1, characterized by the X-ray powder diffraction pattern as shown in FIG. 1.

8. The solid form according to claim 1, characterized by having a melting point with a peak signal at above 215° C., in particular between about 215.6° C. to about 219.6° C., using differential scanning calorimetry with a heating rate of 10 K/min.

9. The solid form according to claim 1,
wherein the solid form is crystalline polymorphic Form A characterized by an IR spectrum comprising at least one peak at one of the positions 689 (±2) cm⁻¹, 1326 (±2) cm⁻¹ or 2874 (±2) cm⁻¹, in particular comprising at least two peaks at positions 689 (±2) cm⁻¹, 1326 (±2) cm⁻¹ or 2874 (±2) cm⁻¹, more particularly comprising the peaks at positions 689 (±2) cm⁻¹, 1326 (±2) cm⁻¹ and 2874 (±2) cm⁻¹.

10. The solid form according to claim 1,
wherein the solid form is crystalline polymorphic Form A characterized by a Raman spectrum comprising at least one peak at one of the positions 691 (±2) cm⁻¹, 1660 (±2) cm⁻¹or 3061 (±2) cm⁻¹, in particular comprising at least two peaks at positions 691 (±2) cm⁻¹, 1660 (±2) cm⁻¹ or 3061 (±2) cm⁻¹, more particularly comprising the peaks at positions 691 (±2) cm⁻¹, 1660 (±2) cm⁻¹ and 3061 (±2) cm⁻¹.

11. A solid form of a compound of formula (I), or a solvate thereof: 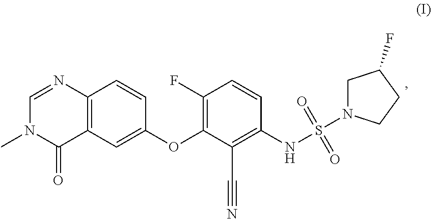 [SMILES: Cn1cnc2ccc(Oc3c(F)ccc(NS(=O)(=O)N4CC[C@@H](F)C4)c3C#N)cc2c1=O]
wherein the solid form is amorphous.

12. A solid form according to claim 11, characterized by exhibiting an onset of a glass transition at a temperature of about 63.1° C. to about 69.1, in particular of about 64.6 to about 67.6, using differential scanning calorimetry with a heating rate of 10 K/min.

13. A solid form according to claim 11, characterized by exhibiting an onset of recrystallization at a temperature of about 114.4° C. to about 120.4° C., in particular between about 115.9° C. to about 118.9° C., using differential scanning calorimetry with a heating rate of 10 K/min.

14. A solid form according to claim 11, wherein the solid form is characterized by an IR spectrum comprising at least one peak at one of the positions 679 (±2) cm⁻¹, 1035 (±2) cm⁻¹ or 1337 (±2) cm⁻¹, in particular comprising at least two peaks at positions 679 (±2) cm⁻¹, 1035 (±2) cm⁻¹ or 1337 (±2) cm⁻¹, more particular comprising the peaks at positions 679 (±2) cm⁻¹, 1035 (±2) cm⁻¹ and 1337 (±2) cm⁻¹.

15. A solid form according to claim 11, wherein the solid form is characterized by a Raman spectrum comprising at least one peak at one of the positions 1159 (±2) cm⁻¹, 1344 (±2) cm⁻¹ or 3069 (±2) cm⁻¹, in particular comprising at least two peaks at positions 1159 (±2) cm⁻¹, 1344 (±2) cm⁻¹ or 3069 (±2) cm⁻¹, more particular having the peaks at positions 1159 (±2) cm⁻¹, 1344 (±2) cm⁻¹ and 3069 (±2) cm⁻¹.

16.-19. (canceled)

20. A pharmaceutical composition comprising a solid form according to claim 1, and one or more pharmaceutically acceptable auxiliary substances.

21.-22. (canceled)

23. A method for therapeutic treatment of a BRAF associated cancer, the method comprising administering a pharmaceutically effective amount of a solid form according to claim 1 to a patient in need thereof.

24. A process for the preparation of a compound of formula (I): 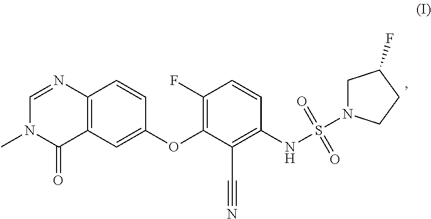 [SMILES: Cn1cnc2ccc(Oc3c(F)ccc(NS(=O)(=O)N4CC[C@@H](F)C4)c3C#N)cc2c1=O]
comprising the following steps:
(a) reacting a compound of formula (A1): 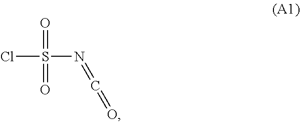
with a compound of formula (A2) 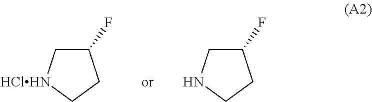
in presence of suitable base in presence of a suitable solvent, to arrive at a compound of formula (B1) 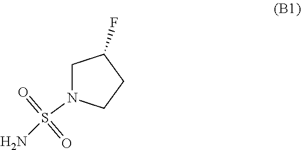
(b) reacting the compound of formula (B1) with a compound of formula (B2): 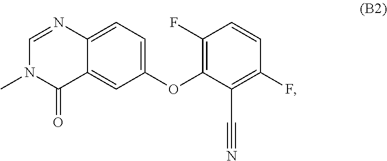 [SMILES: Cn1cnc2ccc(Oc3c(F)ccc(F)c3C#N)cc2c1=O]
in presence of a suitable base in presence of a suitable solvent to arrive at a compound of formula (I): 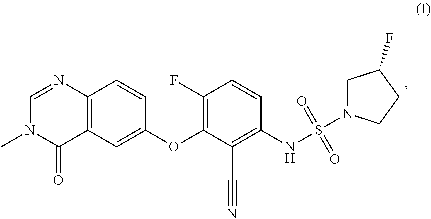 [SMILES: Cn1cnc2ccc(Oc3c(F)ccc(NS(=O)(=O)N4CC[C@@H](F)C4)c3C#N)cc2c1=O]
(c) purifying by filtration a suspension of the crude product of the compound of formula (I) in a suitable solvent and a suitable acid to remove substantial amounts of dimer sulfate and to obtain a filtrate comprising substantial amounts of the compound of formula (I).

25. The process according to claim 24, further comprising at least one of the following steps:
(a) concentrating the filtrate of step (c) comprising substantial amounts of the compound of formula (I) under vacuum to provide a suspension;
(b) adding water and a suitable base to a suspension comprising the compound of formula (I) and after adjustment of the pH to 6.7±0.5 the reaction is stirred to afford a precipitate comprising the compound of formula (I);
(c) filtrating a precipitate comprising the compound of formula (I), followed by washing with a suitable solvent and drying in vacuo to afford crystallized (3R)-N-[2-cyano-4-fluoro-3-(3-methyl-4-oxo-quinazolin-6-yl)oxy-phenyl]-3-fluoro-pyrrolidine-1-sulfonamide;
(d) adding a suitable solvent and water to (3R)-N-[2-cyano-4-fluoro-3-(3-methyl-4-oxo-quinazolin-6-yl)oxy-phenyl]-3-fluoro-pyrrolidine-1-sulfonamide, followed by heating the suspension to between about 40° C. to about 80° C. and filtering and washing the resulting solution with a suitable solvent;
(e) concentrating a solution comprising to (3R)-N-[2-cyano-4-fluoro-3-(3-methyl-4-oxo-quinazolin-6-yl)oxy-phenyl]-3-fluoro-pyrrolidine-1-sulfonamide in a suitable solvent in vacuo;
(f) solvent exchanging a solution comprising (3R)-N-[2-cyano-4-fluoro-3-(3-methyl-4-oxo-quinazolin-6-yl)oxy-phenyl]-3-fluoro-pyrrolidine-1-sulfonamide by distillation and stirring the suspension to precipitate (3R)-N-[2-cyano-4-fluoro-3-(3-methyl-4-oxo-quinazolin-6-yl)oxy-phenyl]-3-fluoro-pyrrolidine-1-sulfonamide; and
(g) filtering and drying a suspension of (3R)-N-[2-cyano-4-fluoro-3-(3-methyl-4-oxo-quinazolin-6-yl)oxy-phenyl]-3-fluoro-pyrrolidine-1-sulfonamide in a suitable solvent in vacuo to afford crystalline polymorphic Form A.

26. (canceled)

27. The method according to claim 23, wherein the BRAF associated cancer is melanoma or non-small cell lung cancer.
